(19) United States
(12) Patent Application Publication
(10) Pub. No.: US 2025/0268636 A1
(43) Pub. Date: Aug. 28, 2025

(54) ORTHOPEDIC SCREW FORMED OF IMPROVED METAL ALLOY

(71) Applicant: MiRus LLC, Marietta, GA (US)

(72) Inventors: Noah Roth, Marietta, GA (US); Jay Yadav, Atlanta, GA (US)

(21) Appl. No.: 19/200,178

(22) Filed: May 6, 2025

Related U.S. Application Data
(63) Continuation-in-part of application No. 29/902,712, filed on Sep. 15, 2023.
(60) Provisional application No. 63/643,031, filed on May 6, 2024.
(60) Provisional application No. 63/785,197, filed on Apr. 8, 2025.

(51) Int. Cl. A61B 17/86

(52) U.S. Cl. CPC A61B 17/8605; A61B 17/8625; A61B 17/866

(57) ABSTRACT

An orthopedic screw that is partially or fully formed of a metal alloy that includes at least 5 awt. % rhenium.

### REFERENCED APPLICATIONS

The present disclosure claims priority on U.S. Provisional Application Ser. No. 63/643,031 filed May 6, 2024, and 63/785,197 filed Apr. 8, 2025, both of which are incorporated fully herein by reference.

The present disclosure is a continuation in part of U.S. application Ser. No. 29/902,712 filed Sep. 15, 2023, which is incorporated fully herein by reference.

### FIELD OF DISCLOSURE

The present disclosure is directed to an orthopedic screw that is formed of an improved metal alloy, and particularly to an orthopedic screw that is partially or fully formed of a rhenium containing metal alloy that has a sufficient quantity of rhenium such that the ductility and tensile strength of the metal alloy is improved.

### BACKGROUND

Bone fractures can be repaired by securing a bone plate across the fracture. The use of a bone plate promotes healing of the fracture by providing a rigid fixation or support structure between the bone and the plate.

Bone plates can be secured to the bone by use of a screw system wherein the screws are locked in the bone plate. A bone screw is generally inserted through an opening in the bone plate and then threaded into the bone. The screw system can optionally be secured to the bone plate by the threads on the head of the screw. Locking a screw into the bone plate can achieve angular and axial stability and eliminate the possibility for the screw to toggle, slide, or be dislodged.

Another type of screw system uses non-locking screws that are secured into bone, but not locked in the screw opening on the bone plate. One advantage of non-locking screws is they can be inserted at various angles because they are not limited by the thread-to-thread contact of locking screw with the bone plate.

There are also bone plates that use both locking and non-locking screws to secure the bone plate to the bone. These types of bone plates typically include a partially threaded opening that can receive either a compression screw or a locking screw.

In view of the current state of the art, it would be desirable to provide a thinner and/or smaller pedicle screw for use with a bone plate without sacrificing the strength of typically larger pedicle screws that are formed of stainless steel or cobalt-chromium alloys.

### SUMMARY OF THE DISCLOSURE

The present disclosure is directed to an orthopedic screw (e.g., bone screw, pedicle screw, etc.) that is formed of an improved metal alloy, and particularly to an orthopedic screw that is partially or fully formed of a rhenium containing metal alloy that has a sufficient quantity of rhenium such that the ductility and tensile strength of the metal alloy is improved. The orthopedic screw can be used with a variety of bone plates that are used for different types of bones such as, but not limited to, spinal bones (e.g., cervical spine (C1-C7), thoracic spine (T1-T12), lumbar spine (L1-L5), sacral spine (S1-S5), tailbone), a radius, an ulna, maxillofacial bones, a fibula, metatarsal bones, calcaneus bones, ankle bones, femur, distal tibia, proximal tibia, proximal humerus, distal humerus, a clavicle, bones of the foot, bones of the hand, etc. As can be appreciated, the orthopedic screw can be used with other types of medical devices (e.g., spinal devices, anchors, implants, etc.).

In one non-limiting aspect of the disclosure, there is provided an orthopedic screw that is partially or fully formed of a metal alloy that includes rhenium. It has been found in some metal alloys, a content of rhenium as low as 5 awt. % can improve one or more properties of the metal alloy that is used to partially or fully form the orthopedic screw. In one one-limiting embodiment, the orthopedic screw is partially (e.g., 0.1-99.999 wt. % and all values and ranges therebetween) or fully formed of a metal alloy that includes at least 5 awt. % rhenium (e.g., 5-99 awt. % [atomic weight] rhenium and all values and ranges therebetween).

In another and/or alternative non-limiting aspect of the disclosure, there is provided an orthopedic screw that is partially or fully formed of a metal alloy that includes rhenium wherein the rhenium content in the metal alloy is in a sufficient quantity as to create a “rhenium effect” in the metal alloy. As defined herein, a “rhenium effect” is a) an increase of at least 10% in ductility of the metal alloy caused by the addition of rhenium to the metal alloy as compared to the same metal alloy that is absent rhenium, and/or b) an increase of at least 10% in tensile strength of the metal alloy caused by the addition of rhenium to the metal alloy as compared to the same metal alloy that is absent rhenium. It has been found for many metal alloys (e.g., stainless steel, CoCr alloys, TiAlV alloys, aluminum alloys, nickel alloys, titanium alloys, tungsten alloys, molybdenum alloys, copper alloys, MP35N alloys, beryllium-copper alloys, etc.), the inclusion of rhenium results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The rhenium effect generally occurs when the atomic weight percent or atomic percent (awt. %) of rhenium in the metal alloy is at least 15 awt. % (e.g., 15-99 awt. % rhenium in the metal alloy and all values and ranges therebetween). For example, for stainless steel alloys, the rhenium effect can begin to be present when the stainless-steel alloy is modified to include a rhenium amount of at least 5-10 wt. % (and all values and ranges therebetween) of the stainless-steel alloy. For CoCr alloys, the rhenium effect can begin to be present when the CoCr alloy is modified to include a rhenium amount of at least 4.8-9.5 wt. % (and all values and ranges therebetween) of the CoCr alloy. For TiAlV alloys, the rhenium effect can begin to be present when the TiAlV alloy is modified to include a rhenium amount of at least 4.5-9 wt. % (and all values and ranges therebetween) of the TiAlV alloy. At can be appreciated, the rhenium content in the above examples can be greater than the minimum amount to create the rhenium effect in the metal alloy.

In accordance with another and/or alternative aspect of the present disclosure, there is provided an orthopedic screw that is partially or fully formed of a metal alloy that includes at least 5 awt. % rhenium, and at least 0.1 wt. % (e.g., 0.1 wt. % to 96 wt. % and all values and ranges therebetween) of one or more metals of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium.

In accordance with another and/or alternative aspect of the present disclosure, there is provided an orthopedic screw that is partially or fully formed of a metal alloy that includes at least 5 awt. % rhenium, and the metal alloy is a refractory metal alloy. As defined herein, a refractory metal alloy is a metal alloy that includes at least 20 wt. % of one or more of molybdenum, rhenium, niobium, tantalum or tungsten. Non-limiting metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative non-limiting aspect of the present disclosure, the orthopedic screw is partially (e.g. 1-99.999 wt. % and all values and ranges therebetween) or fully formed of a metal or metal alloy (e.g., stainless steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal, refractory metal alloy, etc.) that also includes at least 5 atomic weight percent (awt. %) or atomic percent (awt. %) rhenium (e.g., 5-99 awt. % rhenium and all values and ranges therebetween) to form a metal alloy or modified metal alloy. As used herein, atomic weight percent (awt. %) or atomic percent (awt. %) or atomic percentage (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents The terms atomic percentage and atomic weight percentage are used interchangeably and have the same meaning. As defined herein, a stainless-steel alloy (SS alloy) includes at least 50 wt. % (weight percent) iron, 10-28 wt. % chromium, 0-35 wt. % nickel, and optionally one or more of 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-68 wt. % cobalt, 15-32 wt. % chromium, and optionally one or more of 1-38 wt. % nickel, 2-18 wt. % molybdenum, 0-18 wt. % iron, 0-1 wt. % titanium, 0-0.15 wt. % manganese, 0-0.15 wt. % silver, 0-0.25 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox and Elgiloy alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium. As defined herein, a refractory metal alloy is a metal alloy that includes at least 20 wt. % of one or more of molybdenum, rhenium, niobium, tantalum or tungsten. A refractory metal includes niobium, chromium, niobium, molybdenum, rhenium, tantalum, and tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative aspect of the present disclosure, there is provided an orthopedic screw that is partially or fully formed of a metal alloy that includes at least 5 awt. % rhenium, and at least 0.1 wt. % (e.g., 0.1 wt. % to 96 wt. % and all values and ranges therebetween) of one or more of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen, and which metal alloy exhibits a rhenium effect. In one non-limiting embodiment, the metal alloy that is used to partially or fully form the orthopedic screw is a stainless-steel alloy that has been modified to include at least 5 awt. % rhenium. In one non-limiting embodiment, the metal alloy that is used to partially or fully form the orthopedic screw is a cobalt chromium alloy that has been modified to include at least 5 awt. % rhenium. In one non-limiting embodiment, the metal alloy that is used to partially or fully form the orthopedic screw is a TiAlV alloy that has been modified to include at least 5 awt. % rhenium. In one non-limiting embodiment, the metal alloy that is used to partially or fully form the orthopedic screw is an aluminum alloy that has been modified to include at least 5 awt. % rhenium. In one non-limiting embodiment, the metal alloy that is used to partially or fully form the orthopedic screw is a nickel alloy that has been modified to include at least 5 awt. % rhenium. In one non-limiting embodiment, the metal alloy that is used to partially or fully form the orthopedic screw is a titanium alloy that has been modified to include at least 5 awt. % rhenium. In one non-limiting embodiment, the metal alloy that is used to partially or fully form the orthopedic screw is a tungsten alloy that has been modified to include at least 5 awt. % rhenium. In one non-limiting embodiment, the metal alloy that is used to partially or fully form the orthopedic screw is a molybdenum alloy that has been modified to include at least 5 awt. % rhenium. In one non-limiting embodiment, the metal alloy that is used to partially or fully form the orthopedic screw is a copper alloy that has been modified to include at least 5 awt. % rhenium. In one non-limiting embodiment, the metal alloy that is used to partially or fully form the orthopedic screw is a beryllium-copper alloy that has been modified to include at least 5 awt. % rhenium.

In accordance with another and/or alternative aspect of the present disclosure, the metal alloy that is used to partially or fully form the orthopedic screw includes rhenium and molybdenum, and the weight percent of rhenium in the metal alloy is greater that the weight percent of molybdenum in the metal alloy. In one non-limiting embodiment, the metal alloy includes rhenium and molybdenum, and the weight percent of rhenium in the metal alloy is greater that the weight percent of molybdenum in the metal alloy, and the weight percent of one or more of bismuth, niobium, tantalum, tungsten, titanium, vanadium, chromium, manganese, yttrium, zirconium, technetium, ruthenium, rhodium, hafnium, osmium, copper, and iridium in the metal alloy is greater that the weight percent of molybdenum in the metal alloy, and the metal alloy optionally includes 0-2 wt. % of a combination of other metals, carbon, oxygen, phosphorous, sulfur, hydrogen and/or nitrogen.

In accordance with another and/or alternative aspect of the present disclosure, the metal alloy that is used to partially or fully form the orthopedic screw includes rhenium and molybdenum, and the atomic weight percent of rhenium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy that is used to partially or fully form the orthopedic screw includes at least 5 awt. % (e.g., 5-99 awt. % and all values and ranges therebetween) rhenium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another non-limiting embodiment, the metal alloy includes rhenium, molybdenum, and chromium. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, bismuth, calcium, carbon, cobalt, copper, gold, hafnium, iridium, iron, lanthanum, magnesium, manganese, molybdenum, nickel, niobium, osmium, platinum, rare earth metals, rhodium, ruthenium, silver, tantalum, technetium, titanium, tungsten, vanadium, yttrium, zinc, and/or zirconium, and optionally 0-2 wt. % of a combination of other metals, carbon, oxygen, phosphorous, sulfur, hydrogen and/or nitrogen. In another non-limiting embodiment, the metal alloy includes 15-50 awt. % rhenium (and all values and ranges therebetween) and 0.5-70 awt. % chromium (and all values and ranges therebetween). In another non-limiting embodiment, the metal alloy includes 15-50 awt. % rhenium (and all values and ranges therebetween) and 0.5-70 awt. % tantalum (and all values and ranges therebetween). In another non-limiting embodiment, the metal alloy includes 15-50 awt. % rhenium (and all values and ranges therebetween) and 0.5-70 awt. % niobium (and all values and ranges therebetween). In another non-limiting embodiment, the metal alloy includes 15-50 awt. % rhenium (and all values and ranges therebetween) and 0.5-70 awt. % titanium (and all values and ranges therebetween). In another non-limiting embodiment, the metal alloy includes 15-50 awt. % rhenium (and all values and ranges therebetween) and 0.5-70 awt. % zirconium (and all values and ranges therebetween). In another non-limiting embodiment, the metal alloy includes 15-50 awt. % rhenium (and all values and ranges therebetween) and 0.5-70 awt. % molybdenum (and all values and ranges therebetween). In another non-limiting embodiment, the metal alloy includes at least 5 awt. % rhenium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In accordance with another and/or alternative aspect of the present disclosure, the metal alloy that is used to partially or fully form the orthopedic screw includes at least 0.1 wt. % (e.g., 0.1-70 wt. % and all values and ranges therebetween) rhenium and one or more metals selected from the group of molybdenum, chromium, cobalt, nickel, titanium, tantalum, niobium, zirconium, and/or tungsten. In one non-limiting embodiment, the metal alloy that is used to partially or fully form the orthopedic screw includes at least 5 wt. % (e.g., 5-70 wt. % and all values and ranges therebetween) rhenium and one or more metals selected from the group of molybdenum, chromium, cobalt, nickel, titanium, tantalum, niobium, zirconium, and/or tungsten.

In accordance with another and/or alternative aspect of the present disclosure, the metal alloy that is used to partially or fully form the orthopedic screw includes no more than 0.1 wt. % nickel, no more than 0.1 wt. % chromium, and/or no more than 0.1 wt. % cobalt.

In accordance with another and/or alternative aspect of the present disclosure, the metal alloy that is used to partially or fully form the orthopedic screw includes has one or more of the following properties: i) at least 70-100% of the orthopedic screw is formed of a metal alloy that has a yield strength of at least 110 ksi, ii) at least 70-100% of the orthopedic screw is formed of a metal alloy that has a modulus of elasticity of at least 35000 ksi, iii) at least 70-100% of the orthopedic screw is formed of a metal alloy that is formed of a rhenium containing metal alloy that includes at least 0.1 wt. % rhenium and one or more metals selected from the group consisting of Mo, Cr, Co, Ni, Ti, Ta, Nb, Zr, and W.

In accordance with another and/or alternative aspect of the present disclosure, the metal alloy that is used to partially or fully form the orthopedic screw is partially for fully formed of a refractory metal alloy, and wherein the refractory metal alloy includes at least 20 wt. % of one or more of niobium, tantalum or tungsten, and wherein the refractory metal alloy includes 0-30 wt. % molybdenum (and all values and ranges therebetween), and wherein the refractory metal alloy includes at least 5 awt. % rhenium (e.g., 5-80 awt. % rhenium and all values and ranges therebetween).

In accordance with another and/or alternative aspect of the present disclosure, the metal alloy that is used to partially or fully form the orthopedic screw is partially for fully formed of a metal alloy includes at least 5 awt. % rhenium (e.g., 5-99 awt. % rhenium and all values and ranges therebetween), and at least 0.1 wt. % of one or more additive metals selected from aluminum, bismuth, chromium, cobalt, copper, hafnium, iridium, iron, magnesium, manganese, nickel, niobium, osmium, rhodium, ruthenium, silicon, silver, tantalum, technetium, titanium, tungsten, vanadium, yttrium, and zirconium, and wherein the metal alloy includes 0-30 wt. % molybdenum (and all values and ranges therebetween), and wherein a combined weight percent of rhenium and the additive metals is 70-100 wt. % (and all values and ranges therebetween).

In accordance with another and/or alternative aspect of the present disclosure, the metal alloy that is used to partially or fully form the orthopedic screw is partially for fully formed of a metal alloy of stainless steel that has been modified with at least 5 awt. % rhenium (e.g., 5-50 awt. % rhenium and all values and ranges therebetween), and wherein a combined weight percent of iron, chromium, nickel, tantalum, niobium, copper, manganese, aluminum, titanium, selenium, vanadium, tungsten and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of cobalt-chromium alloy that has been modified with at least 5 awt. % rhenium (e.g., 5-50 awt. % rhenium and all values and ranges therebetween), and wherein a combined weight percent of cobalt, chromium, nickel, iron, titanium, manganese, silver, tungsten, silicon, aluminum, iron, boron, silver, titanium, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of titanium-aluminum-vanadium alloy that has been modified with at least 5 awt. % rhenium (e.g., 5-50 awt. % rhenium and all values and ranges therebetween), and wherein a combined weight percent of aluminum, vanadium, titanium, iron, yttrium and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of aluminum alloy that has been modified with at least 5 awt. % rhenium (e.g., 5-50 awt. % rhenium and all values and ranges therebetween), and wherein a combined weight percent of aluminum, silicon, magnesium, manganese, scandium, beryllium, yttrium, cerium, chromium, iron, zinc, titanium, lithium, silver, calcium, zirconium, cadmium, bismuth, nickel, vanadium, gallium, copper, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of nickel alloy that has been modified with at least 5 awt. % rhenium (e.g., 5-50 awt. % rhenium and all values and ranges therebetween), and wherein a combined weight percent of nickel, chromium, iron, copper, cobalt, aluminum, tantalum, tungsten, titanium, niobium, silicon, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of titanium alloy that has been modified with at least 5 awt. % rhenium (e.g., 5-50 awt. % rhenium and all values and ranges therebetween), and wherein a combined weight percent of titanium, aluminum, tin, palladium, vanadium, nickel, ruthenium, chromium, zirconium, niobium, silicon, cobalt, iron, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of tungsten alloy that has been modified with at least 5 awt. % rhenium (e.g., 5-50 awt. % rhenium and all values and ranges therebetween), and wherein a combined weight percent of tungsten, nickel, copper, iron, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of copper alloy that has been modified with at least 5 awt. % rhenium (e.g., 5-50 awt. % rhenium and all values and ranges therebetween), and wherein a combined weight percent of copper, zinc, tin, iron, silicon, manganese, aluminum, beryllium, cobalt, nickel, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of beryllium-copper alloy that has been modified with at least 5 awt. % rhenium (e.g., 5-50 awt. % rhenium and all values and ranges therebetween), and wherein a combined weight percent of copper, beryllium, cobalt, silicon, and rhenium is 70-100 wt. % (and all values and ranges therebetween).

In accordance with another and/or alternative aspect of the present disclosure, the metal alloy that is used to partially or fully form the orthopedic screw is partially for fully formed of a metal alloy of titanium-nickel alloy that has been modified with at least 5 awt. % rhenium (e.g., 5-50 awt. % rhenium and all values and ranges therebetween), and wherein a combined weight percent of nickel, titanium, and rhenium is 70-100 wt. % (and all values and ranges therebetween).

In accordance with another and/or alternative aspect of the present disclosure, the metal alloy includes at least 5 awt. % (e.g., 5-99 awt. % and all values and ranges therebetween) rhenium plus a metal additive that includes at least two metals selected from the group of molybdenum, bismuth, chromium, iridium, niobium, tantalum, titanium, yttrium, and zirconium, and the content of the metal alloy that includes other elements and compounds is 0-0.1 wt. %, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes rhenium, chromium and optionally molybdenum. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more metal additives selected from aluminum, boron, beryllium, bismuth, cadmium, calcium, cerium, chromium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen. In another non-limiting embodiment, the metal alloy includes at least 50 wt. % (e.g., 50-75 wt. % and all values and ranges therebetween) rhenium and at least 25 wt. % chromium (e.g., 25-50 wt. % and all values and ranges therebetween), and optionally 0.1-40 wt. % (and all values and ranges therebetween) of the metal alloy includes one or more metal additives selected from aluminum, boron, beryllium, bismuth, cadmium, calcium, cerium, cobalt, copper, gallium, gold, hafnium, iridium, iron, lanthanum, lanthanum oxide, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen, and wherein a combined weight percent of said rhenium and said chromium is 75-100 wt. % (and all values and ranges therebetween) of the metal alloy. In another non-limiting embodiment, the metal alloy includes at least 10 wt. % (e.g., 10-75 wt. % and all values and ranges therebetween) rhenium and at least 2 wt. % chromium (e.g., 2-50 wt. % and all values and ranges therebetween), at least 10 wt. % molybdenum (e.g., 10-75 wt. % and all values and ranges therebetween), and optionally 0.1-40 wt. % (and all values and ranges therebetween) of the metal alloy includes one or more metal additives selected from aluminum, boron, beryllium, bismuth, cadmium, calcium, cerium, cobalt, copper, gallium, gold, hafnium, iridium, iron, lanthanum, lanthanum oxide, lithium, magnesium, manganese, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen, and wherein a combined weight percent of said rhenium, molybdenum and said chromium is 75-100 wt. % (and all values and ranges therebetween) of the metal alloy.

In accordance with another and/or alternative aspect of the present disclosure, the metal alloy includes a) 15-50 awt. % rhenium (and all values and ranges therebetween), b) 0.5-70 awt. % chromium (and all values and ranges therebetween), and c) 0.1-40 wt. % (and all values and ranges therebetween) of one or more metal additives selected from aluminum, boron, beryllium, bismuth, cadmium, calcium, carbon, cerium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes a) 15-50 awt. % rhenium (and all values and ranges therebetween), b) 0.5-70 awt. % tantalum (and all values and ranges therebetween), and c) 0.1-40 wt. % (and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, technetium, tin, titanium, tungsten, vanadium, yttrium, zinc, and zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes a) 15-50 awt. % rhenium (and all values and ranges therebetween), b) 0.5-70 awt. % niobium (and all values and ranges therebetween), c) 0.1-40 wt. % (and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, osmium, palladium, platinum, rare earth metals, rhodium, ruthenium, scandium, silver, silicon, tantalum, technetium, tin, titanium, tungsten, vanadium, yttrium, zinc, and zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes a) 15-50 awt. % rhenium (and all values and ranges therebetween), b) 0.5-70 awt. % titanium (and all values and ranges therebetween), c) 0.1-40 wt. % (and all values and ranges therebetween) of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, tungsten, vanadium, yttrium, zinc, and zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes a) 15-50 awt. % rhenium (and all values and ranges therebetween), b) 0.5-70 awt. % zirconium (and all values and ranges therebetween), c) 0.1-40 wt. % (and all values and ranges therebetween) of one or more metal additive selected from aluminum, boron, beryllium, bismuth, cadmium, calcium, carbon, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, and zinc, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes a) 15-50 awt. % rhenium (and all values and ranges therebetween), b) 0.5-70 awt. % molybdenum (and all values and ranges therebetween), c) 0.1-40 wt. % (and all values and ranges therebetween) of one or more metal additives selected from aluminum, boron, beryllium, bismuth, cadmium, calcium, carbon, cerium, chromium, cobalt, copper, gallium, gold, hafnium, iridium, iron, lanthanum, lithium, magnesium, manganese, nickel, niobium, osmium, palladium, platinum, rare earth metals, rhodium, ruthenium, scandium, silver, silicon, tantalum, technetium, tin, titanium, tungsten, vanadium, yttrium, zinc, and zirconium, and wherein the metal alloy optionally includes 0-2 wt. % (and all values and ranges therebetween) of a combination of metals other than the metal additives, carbon, oxygen, phosphorous, sulfur, hydrogen and/or nitrogen.

In accordance with another and/or alternative aspect of the present disclosure, the metal alloy includes a) at least 15 awt. % rhenium, b) greater than 50 wt. % titanium (e.g., 51-80 wt. % and all values and ranges therebetween), c) 15-45 wt. % (and all values and ranges therebetween) niobium, d) 0-10 wt. % (and all values and ranges therebetween) zirconium, e) 0-15 wt. % (and all values and ranges therebetween) tantalum, and f) and 0-8 wt. % molybdenum (and all values and ranges therebetween), and wherein the metal alloy optionally includes 0-2 wt. % (and all values and ranges therebetween) of a combination of metals other than the rhenium, titanium, niobium, zirconium, tantalum, molybdenum, carbon, oxygen, phosphorous, sulfur, hydrogen and/or nitrogen.

In accordance with another and/or alternative aspect of the present disclosure, the metal alloy that is used to partially or fully form the orthopedic screw includes 35-75 wt. % (e.g., and all values and ranges therebetween) of the metal alloy includes rhenium, and 25-65 wt. % (and all values and ranges therebetween) of the metal alloy includes two or more of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy includes 0-2 wt. % (and all values and ranges therebetween) of a combination of other combination of other metals, carbon, oxygen, phosphorous, sulfur, hydrogen and nitrogen.

In accordance with another and/or alternative aspect of the present disclosure, the metal alloy includes rhenium and molybdenum, and the weight percent of rhenium in the metal alloy is greater that the weight percent of molybdenum in the metal alloy. In one non-limiting embodiment, the metal alloy includes rhenium and molybdenum, and the weight percent of rhenium in the metal alloy is greater that the weight percent of molybdenum in the metal alloy, and the weight percent of one or more of bismuth, niobium, tantalum, tungsten, titanium, vanadium, chromium, manganese, yttrium, zirconium, technetium, ruthenium, rhodium, hafnium, osmium, copper, and iridium in the metal alloy is greater that the weight percent of molybdenum in the metal alloy. In another non-limiting embodiment, the metal alloy includes rhenium and molybdenum, and the weight percent of rhenium in the metal alloy is greater that the weight percent of molybdenum in the metal alloy, and the weight percent of one or more of bismuth, niobium, tantalum, tungsten, titanium, vanadium, chromium, manganese, yttrium, zirconium, technetium, ruthenium, rhodium, hafnium, osmium, copper, and iridium in the metal alloy is greater that the weight percent of molybdenum in the metal alloy, and the weight percent of molybdenum in the metal alloy is 0.1-15 wt. % (and all values and ranges therebetween). In another non-limiting embodiment, the metal alloy includes rhenium and molybdenum, and the weight percent of rhenium in the metal alloy is greater that the weight percent of molybdenum in the metal alloy, and the weight percent of one or more of bismuth, niobium, tantalum, tungsten, titanium, vanadium, chromium, manganese, yttrium, zirconium, technetium, ruthenium, rhodium, hafnium, osmium, copper, and iridium in the metal alloy is greater that the weight percent of molybdenum in the metal alloy, and the weight percent of molybdenum in the metal alloy is 0.1-15 wt. %, and the metal alloy includes 0-2 wt. % (and all values and ranges therebetween) of a combination of other metals, carbon, oxygen, phosphorous, sulfur, hydrogen and nitrogen.

In accordance with another and/or alternative aspect of the present disclosure, the metal alloy includes rhenium and molybdenum, and the atomic weight percent of rhenium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium is 0.7:1 to 1.5:1 (and all values and ranges therebetween).

In accordance with another and/or alternative aspect of the present disclosure, the metal alloy includes rhenium and molybdenum, and the metal alloy includes at least 5 awt. % rhenium and two additional metals selected from bismuth, niobium, tantalum, tungsten, titanium, vanadium, chromium, manganese, yttrium, zirconium, technetium, ruthenium, rhodium, hafnium, osmium, copper, and iridium, the atomic ratio of the two additional metals is 0.4:1 to 2.5:1 (and all values and ranges therebetween).

In accordance with another and/or alternative aspect of the present disclosure, the metal alloy includes 15-60 awt. % rhenium and one or more metals selected from the group consisting of 0.5-70 awt. % chromium (and all values and ranges therebetween), 0.5-70 awt. % tantalum (and all values and ranges therebetween), 0.5-70 at. % niobium (and all values and ranges therebetween), 0.5-70 awt. % titanium (and all values and ranges therebetween), 0.5-70 awt. % zirconium (and all values and ranges therebetween), and 0.5-70 awt. % molybdenum (and all values and ranges therebetween).

In accordance with another and/or alternative aspect of the present disclosure, the metal alloy includes 15-50 awt. % rhenium (and all values and ranges therebetween) and 0.5-70 awt. % chromium (and all values and ranges therebetween).

In accordance with another and/or alternative aspect of the present disclosure, the metal alloy includes 15-50 awt. % rhenium (and all values and ranges therebetween) and 0.5-70 awt. % tantalum (and all values and ranges therebetween).

In accordance with another and/or alternative aspect of the present disclosure, the metal alloy includes 15-50 awt. % rhenium (and all values and ranges therebetween) and 0.5-70 awt. % niobium (and all values and ranges therebetween).

In accordance with another and/or alternative aspect of the present disclosure, the metal alloy includes 15-50 awt. % rhenium (and all values and ranges therebetween) and 0.5-70 awt. % titanium (and all values and ranges therebetween).

In accordance with another and/or alternative aspect of the present disclosure, the metal alloy includes 15-50 awt. % rhenium (and all values and ranges therebetween) and 0.5-70 awt. % zirconium (and all values and ranges therebetween).

In accordance with another and/or alternative aspect of the present disclosure, the metal alloy includes at least 5 awt. % rhenium, greater than 50 wt. % titanium (e.g., 51-80 wt. % and all values and ranges therebetween), 15-45 wt. % (and all values and ranges therebetween) niobium, 1-10% wt. % (and all values and ranges therebetween) zirconium, and 1-15 wt. % (and all values and ranges therebetween) tantalum.

In accordance with another and/or alternative aspect of the present disclosure, the metal alloy includes at least 5 awt. % rhenium, greater than 50 wt. % titanium (e.g., 51-80 wt. % and all values and ranges therebetween), 15-45 wt. % (and all values and ranges therebetween) niobium, and 1-10% wt. % (and all values and ranges therebetween) molybdenum.

In accordance with another and/or alternative aspect of the present disclosure, the metal alloy includes at least 5 awt. % rhenium, 30-60 wt. % cobalt (and all values and ranges therebetween), 10-30 wt. % chromium (and all values and ranges therebetween), 5-20 wt. % iron (and all values and ranges therebetween), 5-22 wt. % nickel (and all values and ranges therebetween), and 2-12 wt. % molybdenum (and all values and ranges therebetween).

In accordance with another and/or alternative aspect of the present disclosure, the metal alloy includes at least 5 awt. % rhenium, 30-60 wt. % zirconium (and all values and ranges therebetween), and 30-60 wt. % molybdenum (and all values and ranges therebetween).

In accordance with another and/or alternative aspect of the present disclosure, the metal alloy includes at least 5 awt. % rhenium, 80-95 wt. % niobium (and all values and ranges therebetween), and 0.5-10 wt. % zirconium (and all values and ranges therebetween).

In accordance with another and/or alternative aspect of the present disclosure, the metal alloy includes at least 5 awt. % rhenium, 55-75 wt. % niobium (and all values and ranges therebetween), 18-40 wt. % tantalum (and all values and ranges therebetween), 1-7 wt. % tungsten (and all values and ranges therebetween), and 0.5-4 wt. % zirconium (and all values and ranges therebetween).

In accordance with another and/or alternative aspect of the present disclosure, the metal alloy that is used to partially or fully form the orthopedic screw includes rhenium plus at least one additive selected form the group of aluminum, bismuth, calcium, chromium, cobalt, copper, gold, hafnium, iridium, iron, lanthanum, lead, magnesium, manganese, molybdenum, nickel, niobium, osmium, platinum, rare earth metals, rhodium, ruthenium, silver, silicon, tantalum, technetium, tin, titanium, tungsten, vanadium, yttrium, zinc, and/or zirconium, and less than about 5 wt. % (e.g., 0-4.999999 wt. % and all values and ranges therebetween) of other metals and/or impurities. A high purity level of the metal alloy results in the formation of a more homogeneous alloy, which in turn results in a more uniform density throughout the metal alloy, and also results in the desired yield and ultimate tensile strengths of the metal alloy.

In accordance with another and/or alternative aspect of the present disclosure, the metal alloy that is used to partially of fully form the orthopedic screw optionally has one or more of the following properties: i) has a yield strength of at least 110 ksi (e.g., 100-275 ksi and all values and ranges therebetween), ii) has a modulus of elasticity of at least 35000 ksi, and/or iii) a Moh's hardness of greater than 6 (e.g., 6.05-9.8 and all values and ranges therebetween). In one non-limiting embodiment, the average Vickers hardness of the metal alloy is optionally at least about 150 Vickers (e.g., 150-300 Vickers and all values and ranges therebetween), and typically 160-240 Vickers; however, this is not required. In another and/or alternative non-limiting embodiment of the disclosure, the average ultimate tensile strength of the metal alloy is optionally at least about 125 ksi (e.g., 125-300 ksi and all values and ranges therebetween); however, this is not required. In another and/or alternative non-limiting embodiment of the disclosure, the average grain size of the metal alloy is optionally no greater than about 4 ASTM (e.g., 4 ASTM to 20 ASTM using ASTM E112 and all values and ranges therebetween, e.g., 0.35 micron to 90 micron, and all values and ranges therebetween). In another and/or alternative non-limiting embodiment of the disclosure, the average tensile elongation of the metal alloy is optionally at least about 25% (e.g., 25-50% average tensile elongation and all values and ranges therebetween).

In accordance with another and/or alternative aspect of the present disclosure, there is provided an orthopedic screw that can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.).

The metal alloy used to at least partially form the orthopedic screw can result in improved properties as compared to similarly shaped and size orthopedic screws that are formed of stainless steel, cobalt-chromium alloy, and titanium alloys (e.g., TiAlV) such as 1) increasing the radiopacity of the orthopedic screw, 2) increasing the yield strength and/or ultimate tensile strength of the orthopedic screw, 3) improving the stress-strain properties of the orthopedic screw, 4) improving the bendability and/or flexibility of the orthopedic screw, 5) improving the strength and/or durability of the orthopedic screw, 6) increasing the hardness of the orthopedic screw, 7) improving the biostability and/or biocompatibility properties of the orthopedic screw, 8) increasing the fatigue resistance of the orthopedic screw, 9) resisting cracking in the orthopedic screw and resist propagation of crack, 10) enabling a smaller, thinner, and/or lighter weight orthopedic screw to be made, 11) improving fatigue ductility of the orthopedic screw, 12) improving durability of the orthopedic screw, 13) improving the fatigue life of the orthopedic screw, 14) reducing adverse tissue reactions after implantation of the orthopedic screw, 15) reducing metal ion release after implantation of the orthopedic screw, 16) reducing corrosion of the orthopedic screw after implantation of the orthopedic screw, 17) reducing allergic reaction after implantation of the orthopedic screw, 18) improving hydrophilicity of the orthopedic screw, 19) reducing the thickness of a portion or all of the metal component of the orthopedic screw, 20) improving bone fusion with the orthopedic screw, 21) lowering ion release from the orthopedic screw into tissue after implantation of the orthopedic screw, 22) reducing magnetic susceptibility of the orthopedic screw when implanted in a patient, and/or 23) reducing toxicity of the orthopedic screw after implantation of the orthopedic screw. These one or more improved physical properties of the metal alloy can be achieved in the orthopedic screw without having to increase the bulk, volume, and/or weight of the orthopedic screw as compared to similarly shaped orthopedic screws that are partially or fully formed from stainless steel, titanium alloy, or cobalt-chromium alloy and, in some instances, these improved physical properties can be obtained even when the volume, bulk, and/or weight of the orthopedic screw is reduced as compared to similarly shaped orthopedic screws that are at least partially formed from stainless steel, titanium alloy, or cobalt-chromium alloys.

In accordance with another and/or alternative aspect of the present disclosure, the orthopedic screw can be subjected to one or more manufacturing processes such as expansion, laser cutting, etching, crimping, annealing, drawing, pilgering, electroplating, electro-polishing, machining, plasma coating, 3D printed coatings, chemical vapor deposition, chemical polishing, cleaning, pickling, ion beam deposition or implantation, sputter coating, vacuum deposition, etc.

In accordance with another and/or alternative aspect of the present disclosure, the metal alloy that is used to partially or fully form the orthopedic screw optionally includes a certain amount of carbon and oxygen; however, this is not required. These two elements have been found to affect the forming properties and brittleness of the metal alloy. The controlled atomic ratio of carbon and oxygen of the metal alloy also minimize the tendency of the metal alloy to form micro-cracks during the forming of the metal alloy at least partially into an orthopedic screw, and/or during the use of the orthopedic screw in a body. The control of the atomic ratio of carbon to oxygen in the metal alloy allows for the redistribution of oxygen in the metal alloy to minimize the tendency of micro-cracking in the metal alloy during the forming of the metal alloy at least partially into an orthopedic screw, and/or during the use of the orthopedic screw in a body. The atomic ratio of carbon to oxygen in the metal alloy is believed to facilitate in minimizing the tendency of micro-cracking in the metal alloy and improve the degree of elongation of the metal alloy, both of which can affect one or more physical properties of the metal alloy that are useful or desired in forming and/or using the medical device. The carbon to oxygen atomic ratio can be as low as about 0.2:1 (e.g., 0.2:1 to 50:1 and all values and ranges therebetween). Typically, the carbon content of the metal alloy is less than about 0.1 wt. % (e.g., 0-0.0999999 wt. % and all values and ranges therebetween). Generally, the oxygen content is less than about 0.1 wt. % of the metal alloy (e.g., 0-0.0999999 wt. % and all values and ranges therebetween).

In accordance with another and/or alternative aspect of the present disclosure, the metal alloy that is used to partially or fully form the orthopedic screw optionally includes a controlled amount of nitrogen; however, this is not required. Large amounts of nitrogen in the metal alloy can adversely affect the ductility of the metal alloy. This can in turn adversely affect the elongation properties of the metal alloy. A too high nitrogen content in the metal alloy can begin to cause the ductility of the metal alloy to unacceptably decrease, thus adversely affect one or more physical properties of the metal alloy that are useful or desired in forming and/or using the orthopedic screw. In one non-limiting formulation, the metal alloy includes less than about 0.001 wt. % nitrogen (e.g., 0 wt. % to 0.0009999 wt. % and all values and ranges therebetween). It is believed that the nitrogen content should be less than the content of carbon or oxygen in the metal alloy. In one non-limiting formulation, the atomic ratio of carbon to nitrogen is at least about 1.5:1 (e.g., 1.5:1 to 400:1 and all values and ranges therebetween).

In accordance with another and/or alternative aspect of the present disclosure, the orthopedic screw includes at least about 5 wt. % of a metal alloy (e.g., 5-100 wt. % and all values and ranges therebetween) that includes at least 5 awt. % rhenium.

In accordance with another and/or alternative aspect of the present disclosure, the metal alloy that includes at least 5 awt. % rhenium and that is used to form all or part of the orthopedic screw 1) is not clad, metal sprayed, plated and/or formed (e.g., cold worked, hot worked, etc.) onto another metal, or 2) does not have another metal or metal alloy metal sprayed, plated, clad and/or formed onto the novel metal alloy.

In accordance with another and/or alternative aspect of the present disclosure, the metal alloy that includes at least 5 awt. % rhenium and that is used to form all or part of the orthopedic screw can be used to form a) a coating on a portion of all of the orthopedic screw, or b) a core of a portion or all of the orthopedic screw. The coating thickness of the metal alloy is non-limiting (e.g., 1 μm to 1 inch and all values and ranges therebetween). In one non-limiting example, there is provided an orthopedic screw wherein the core of the orthopedic screw is formed of a metal or metal alloy (e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, metal alloy that includes at least 5 awt. % rhenium, etc.) or ceramic or composite material, and the other layer of the clad orthopedic screw is formed of the a metal or metal alloy that has a different composition from the core (e.g., chromium alloy, titanium, titanium alloy, stainless steel, iron alloy, CoCr alloy, rhenium alloy, molybdenum alloy, tungsten alloy, Ta—W alloy, refractory metal alloy, MoTa alloy, MoRe alloy, metal alloy that includes at least 5 awt. % rhenium, etc.). The core and the other layer of the orthopedic screw can each form 10-99% (and all values and ranges therebetween) of the overall cross section of the orthopedic screw. In one non-limiting embodiment, the outer layer of the orthopedic screw is a metal alloy that includes at least 5 awt. % rhenium. The base hardness of the metal alloy that includes at least 5 awt. % rhenium can be as low as 300 Vickers and/or as high as 500 Vickers (and all values and ranges therebetween). In instances where the properties of fully annealed material are desired, but only the surface requires to be hardened, the present disclosure includes a method that can provide benefits of both a softer metal alloy with a harder outer surface or shell. A non-limiting example is an orthopedic screw where a softer iron alloy is desired for high ductility as well as ease of machinability. Simultaneously, a hard shell is desired for a finished orthopedic screw. While the inner hardness can range from 250 Vickers to 550 Vickers (and all values and ranges therebetween), the outer hardness can vary from 350 Vickers to 1000 Vickers (and all values and ranges therebetween) when using a metal alloy that includes at least 5 awt. % rhenium. As can be appreciated, other inner and outer hardness values can be used for the orthopedic screw.

In accordance with another and/or alternative aspect of the present disclosure, the orthopedic screw can optionally be formed from a tube or rod, or be formed into a shape that is at least 80% of the final net shape of the orthopedic screw.

In accordance with another and/or alternative aspect of the present disclosure, the metal alloy that includes at least 5 awt. % rhenium and is used to partially or fully form the orthopedic screw is optionally subjected to a swaging process. In one non-limiting embodiment, swaging is performed on the orthopedic screw to at least partially or fully achieve final dimensions of one or more portions of the orthopedic screw. Swaging temperatures for a particular metal alloy can vary. For a metal alloy that includes at least 5 awt. % rhenium, the swaging temperature can be from room temperature (RT) (e.g., 10-27° C. and all values and ranges therebetween) to about 400° C. (e.g., 10-400° C. and all values and ranges therebetween) if the swaging is conducted in air or an oxidizing environment. The swaging temperature can be increased to up to about 1500° C. (e.g., 10-15% 00° C. and all values and ranges therebetween) if the swaging process is performed in a controlled neutral or non-reducing environment (e.g., inert environment). The swaging process can be conducted by repeatedly hammering the orthopedic screw at the location to be hardened at the desired swaging temperature. In one non-limiting embodiment, during the swaging process ions of boron and/or nitrogen are allowed to impinge upon rhenium atoms in the metal alloy that includes at least 5 awt. % rhenium so as to form ReB₂, ReN₂ and/or ReN₃; however, this is not required. It has been found that ReB₂, ReN₂ and/or ReN₃ are ultra-hard compounds. In one non-limiting process, the orthopedic screw can be machined and shape when the metal alloy that includes at least 5 awt. % rhenium is in a less hardened state. As such, the metal alloy that includes at least 5 awt. % rhenium can be first annealed to soften and then machined into a desired shape. After the metal alloy that includes at least 5 awt. % rhenium is shaped, the metal alloy that includes at least 5 awt. % rhenium can be re-hardened. The hardening of the metal alloy that includes at least 5 awt. % rhenium improves the wear resistance and/or shape retention of the orthopedic screw. The metal alloy that includes at least 5 awt. % rhenium generally cannot be re-hardened by annealing, thus a special rehardening processes is required. Such rehardening can be achieved by the swaging process of the present disclosure.

In accordance with another and/or alternative aspect of the present disclosure, the metal alloy can optionally be nitrided; however, this is not required. The nitrided layer on the metal alloy can function as a lubricating surface during the optional drawing of the metal alloy when partially or fully forming the orthopedic screw. After the metal alloy is nitrided, the metal alloy is typically cleaned; however, this is not required. The thickness of the nitrided surface layer is less than about 1 mm. In one non-limiting embodiment, the thickness of the nitride surface layer is at least about 50 nanometer and less than about 1 mm (and all values and ranges therebetween). Generally, the weight percent of nitrogen in the nitrided surface layer is 0.0001-5 wt. % nitrogen (and all values and ranges therebetween). In one non-limiting composition of the nitrided surface layer on a metal alloy that includes at least 5 awt. % rhenium comprises rhenium and 0.0001-5 wt. % nitrogen (and all values and ranges therebetween). The nitriding process for the metal alloy can be used to increase surface hardness and/or wear resistance of the orthopedic screw, and/or to inhibit or prevent discoloration of the metal alloy (e.g., discoloration by oxidation, etc.).

In accordance with another and/or alternative aspect of the present disclosure, the orthopedic screw can optionally contain and/or be coated with one or more agents that facilitate in the success of the orthopedic screw and/or treated area. The term “agent” includes, but is not limited to, a substance, pharmaceutical, biologic, veterinary product, drug, and analogs or derivatives otherwise formulated and/or designed to prevent, inhibit, and/or treat one or more clinical and/or biological events, and/or to promote healing. Non-limiting examples of clinical events that can be addressed by one or more agents include, but are not limited to, viral, fungus and/or bacterial infection; vascular diseases and/or disorders, digestive diseases and/or disorders, reproductive diseases and/or disorders, lymphatic diseases and/or disorders, cancer, implant rejection, pain, nausea, swelling, arthritis, bone diseases and/or disorders, organ failure, immunity diseases and/or disorders, cholesterol problems, blood diseases and/or disorders, lung diseases and/or disorders, heart diseases and/or disorders, brain diseases and/or disorders, neuralgia diseases and/or disorders, kidney diseases and/or disorders, ulcers, liver diseases and/or disorders, intestinal diseases and/or disorders, gallbladder diseases and/or disorders, pancreatic diseases and/or disorders, psychological disorders, respiratory diseases and/or disorders, gland diseases and/or disorders, skin diseases and/or disorders, hearing diseases and/or disorders, oral diseases and/or disorders, nasal diseases and/or disorders, eye diseases and/or disorders, fatigue, genetic diseases and/or disorders, burns, scarring and/or scars, trauma, weight diseases and/or disorders, addiction diseases and/or disorders, hair loss, cramp, muscle spasms, tissue repair, nerve repair, neural regeneration, and/or the like. The type and/or amount of agent included in the orthopedic screw and/or coated on orthopedic screw can vary. When two or more agents are included in and/or coated on the orthopedic screw, the amount of the two or more agents can be the same or different. The one or more agents can be coated on and/or impregnated in the orthopedic screw by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the disclosure, the type and/or amount of agent included on, in, and/or in conjunction with the orthopedic screw is generally selected for the treatment of one or more medical treatments. The amount of two or more agents on, in, and/or used in conjunction with the orthopedic screw can be the same or different. The one or more agents, when used on and/or in the orthopedic screw, can optionally be released in a controlled manner so the area in question to be treated is provided with the desired dosage of agent over a sustained period of time. As can be appreciated, controlled release of one or more agents on the orthopedic screw is not always required and/or desirable. As such, one or more of the agents on and/or in the orthopedic screw can be uncontrollably released from the orthopedic screw during and/or after insertion of the orthopedic screw in the treatment area. It can also be appreciated that one or more agents on and/or in the orthopedic screw can be controllably released from the orthopedic screw and one or more agents on and/or in the orthopedic screw can be uncontrollably released from the orthopedic screw. It can also be appreciated that one or more agents on and/or in one region of the orthopedic screw can be controllably released from the orthopedic screw and one or more agents on and/or in the orthopedic screw can be uncontrollably released from another region on the orthopedic screw. As such, the orthopedic screw can be designed such that 1) all the agent on and/or in the orthopedic screw is controllably released, 2) some of the agent on and/or in the orthopedic screw is controllably released and some of the agent on the orthopedic screw is non-controllably released, or 3) none of the agent on and/or in the orthopedic screw is controllably released. The orthopedic screw can also be designed such that the rate of release of the one or more agents from the orthopedic screw is the same or different. The orthopedic screw can also be designed such that the rate of release of the one or more agents from one or more regions on the orthopedic screw is the same or different. Non-limiting arrangements that can be used to control the release of one or more agents from the orthopedic screw include 1) at least partially coating one or more agents with one or more polymers, 2) at least partially incorporating and/or at least partially encapsulating one or more agents into and/or with one or more polymers, and/or 3) inserting one or more agents in pores, passageway, cavities, etc., in the orthopedic screw and at least partially coat or cover such pores, passageway, cavities, etc., with one or more polymers. As can be appreciated, other or additional arrangements can be used to control the release of one or more agents from the orthopedic screw. The one or more polymers, when used to at least partially control the release of one or more agents from the orthopedic screw, can be porous or non-porous. The one or more agents can be inserted into and/or applied to one or more surface structures and/or micro-structures on the orthopedic screw, and/or be used to at least partially form one or more surface structures and/or micro-structures on the orthopedic screw. As such, the one or more agents on the orthopedic screw can be 1) coated on one or more surface regions of the orthopedic screw, 2) inserted and/or impregnated in one or more surface structures and/or micro-structures, etc., of the orthopedic screw, and/or 3) form at least a portion or be included in at least a portion of the structure of the orthopedic screw. When the one or more agents are coated on the orthopedic screw, the one or more agents can 1) be directly coated on one or more surfaces of the orthopedic screw, 2) be mixed with one or more coating polymers or other coating materials and then at least partially coated on one or more surfaces of the orthopedic screw, 3) be at least partially coated on the surface of another coating material that has been at least partially coated on the orthopedic screw, and/or 4) be at least partially encapsulated between a) a surface or region of the orthopedic screw and one or more other coating materials and/or b) two or more other coating materials. As can be appreciated, many other coating arrangements can be additionally or alternatively used. When the one or more agents are optionally inserted and/or impregnated in one or more internal structures, surface structures and/or micro-structures of the orthopedic screw, 1) one or more other coating materials can be applied at least partially over the one or more internal structures, surface structures, and/or micro-structures of the orthopedic screw, and/or 2) one or more polymers can be combined with one or more agents. As such, the one or more agents can be 1) embedded in the structure of the orthopedic screw, 2) positioned in one or more internal structures of the orthopedic screw, 3) encapsulated between two polymer coatings, 4) encapsulated between the base structure and a polymer coating, 5) mixed in the base structure of the orthopedic screw that includes at least one polymer coating, or 6) one or more combinations of 1, 2, 3, 4, and/or 5. In addition or alternatively, the one or more coating of the one or more polymers on the orthopedic screw can include 1) one or more coatings of non-porous polymers, 2) one or more coatings of a combination of one or more porous polymers and one or more non-porous polymers, 3) one or more coating of porous polymer, or 4) one or more combinations of options 1, 2, and 3.

In another and/or alternative aspect of the present disclosure, different agents can optionally be located in and/or between different polymer coating layers and/or on the structure of the orthopedic screw. As can also be appreciated, many other and/or additional coating combinations and/or configurations can be used. The concentration of one or more agents, the type of polymer, the type and/or shape of internal structures in the orthopedic screw, and/or the coating thickness of one or more agents can be used to control the release time, the release rate, and/or the dosage amount of one or more agents; however, other or additional combinations can be used. As such, the agent and polymer system combination and location on the orthopedic screw can be numerous. As can also be appreciated, one or more agents can be deposited on the top surface of the orthopedic screw to provide an initial uncontrolled burst effect of the one or more agents prior to the 1) controlled release of the one or more agents through one or more layers of a polymer system that include one or more non-porous polymers, and/or 2) uncontrolled release of the one or more agents through one or more layers of a polymer system. The one or more agents and/or polymers can be coated on the orthopedic screw by a variety of mechanisms such as, but not limited to, spraying (e.g., atomizing spray techniques, etc.), dip coating, roll coating, sonication, brushing, plasma deposition, and/or depositing by vapor deposition.

In another and/or alternative aspect of the present disclosure, a variety of polymers can optionally be coated on the orthopedic screw and/or be used to form at least a portion of the orthopedic screw. The one or more polymers can be used on the orthopedic screw for a variety of reasons such as, but not limited to, 1) forming a portion of the orthopedic screw, 2) improving a physical property of the orthopedic screw (e.g., improve strength, improve durability, improve biocompatibility, reduce friction, etc.), 3) forming a protective coating on one or more surface structures on the orthopedic screw, 4) at least partially forming one or more surface structures on the orthopedic screw, and/or 5) at least partially controlling a release rate of one or more agents from the orthopedic screw. As can be appreciated, the one or more polymers can have other or additional uses on the orthopedic screw. The one or more polymers can be porous, non-porous, biostable, biodegradable (i.e., dissolves, degrades, is absorbed, or any combination thereof in the body), and/or biocompatible. When the orthopedic screw is coated with one or more polymers, the polymer can include 1) one or more coatings of non-porous polymers, 2) one or more coatings of a combination of one or more porous polymers and one or more non-porous polymers, 3) one or more coatings of one or more porous polymers and one or more coatings of one or more non-porous polymers, 4) one or more coating of porous polymer, or 5) one or more combinations of options 1, 2, 3, and 4. The thickness of one or more of the polymer layers can be the same or different. When one or more layers of polymer are coated onto at least a portion of the orthopedic screw, the one or more coatings can be applied by a variety of techniques such as, but not limited to, vapor deposition and/or plasma deposition, spraying, dip-coating, roll coating, sonication, atomization, brushing, and/or the like; however, other or additional coating techniques can be used. The one or more polymers that can be coated on the orthopedic screw and/or used to at least partially form the orthopedic screw can be polymers that are considered to be biodegradable, bioresorbable, or bioerodable; polymers that are considered to be biostable; and/or polymers that can be made to be biodegradable and/or bioresorbable with modification. The thickness of each polymer layer is generally at least about 0.01 μm and is generally less than about 150 μm (e.g., 0.01 μm to 150 μm and all values and ranges therebetween); however, other thicknesses can be used. In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In accordance with another and/or alternative aspect of the present disclosure, the orthopedic screw, when including and/or is coated with one or more agents, can include and/or can be coated with one or more agents that are the same or different in different regions of the orthopedic screw and/or have differing amounts and/or concentrations in differing regions of the orthopedic screw. For instance, the orthopedic screw can 1) be coated with and/or include one or more biologicals on at least one portion of the orthopedic screw and at least another portion of the orthopedic screw is not coated with and/or includes agent; 2) be coated with and/or include one or more biologicals on at least one portion of the orthopedic screw that is different from one or more biologicals on at least another portion of the orthopedic screw; and/or 3) be coated with and/or include one or more biologicals at a concentration on at least one portion of the orthopedic screw that is different from the concentration of one or more biologicals on at least another portion of the orthopedic screw.

In accordance with another and/or alternative aspect of the present disclosure, one or more portions of the orthopedic screw can optionally 1) include the same or different agents, 2) include the same or different amount of one or more agents, 3) include the same or different polymer coatings, 4) include the same or different coating thicknesses of one or more polymer coatings, 5) have one or more portions of the orthopedic screw controllably release and/or uncontrollably release one or more agents, and/or 6) have one or more portions of the orthopedic screw controllably release one or more agents and one or more portions of the orthopedic screw uncontrollably release one or more agents.

In accordance with another and/or alternative aspect of the present disclosure, one or more surfaces of the orthopedic screw can optionally be treated to achieve the desired coating properties of the one or more agents and one or more polymers coated on the orthopedic screw. Such surface treatment techniques include, but are not limited to, cleaning, buffing, smoothing, nitriding, annealing, swaging, cold working, etching (chemical etching, plasma etching, etc.), etc. As can be appreciated, other or additional surface treatment processes can be used prior to the coating of one or more agents and/or polymers on the surface of the orthopedic screw.

In another and/or alternative non-limiting aspect of the present disclosure, there is provided an orthopedic screw that is at least partially (e.g. 1-99.999 wt. % and all values and ranges therebetween) or fully formed of a material that is partially of fully coated with an enhancement layer used to a) reduced metal ion release of the metal material from the orthopedic screw, and/or b) reduce the rate of corrosion on the metal that forms the orthopedic screw, and/or promote healing about the orthopedic screw. In non-limiting embodiment, the enhancement layer on one or more portions of the orthopedic screw is formulated to provide and/or promote generation of nitric oxide near, at and/or in adjacent tissue. In one non-limiting arrangement, there is provided a metal oxynitride layer that is deposited a portion or all of the orthopedic screw. In one non-limiting specific configuration, the metal oxynitride layer is or includes titanium oxynitride and/or zirconium oxynitride. In another non-limiting specific configuration, the thickness of the metal oxynitride layer is at least 10 nanometers (e.g., 10 nanometers to 10 microns and all values and ranges therebetween). In one non-limiting specific configuration, the oxygen to nitrogen atomic ratios of the metal oxynitride layer is 1:10 to 10:1 (and all values and ranges therebetween). In another non-limiting specific configuration, the layer of metal oxynitride layer is optionally deposited onto a metallic adhesion layer in between the outer surface of the orthopedic screw and the oxynitride layer, and wherein the adhesion layer optionally is or includes titanium metal and/or zirconium metal, and wherein the adhesion layer optionally has a thickness of 10 nanometers (e.g., 10 to 500 nanometers and all values and ranges therebetween). The enhancement layer and/or the metallic adhesion layer can be applied by use of a vacuum coating process (e.g., physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process), plating process, etc. When one or more agents are coated on the orthopedic screw, and the orthopedic screw includes an enhancement layer, one or more agents are generally coated on the outer surface of the enhancement layer. As defined herein, an agent is not an enhancement coating or layer.

In another and/or alternative non-limiting aspect of the disclosure, the orthopedic screw can optionally include a marker material that facilitates enabling the orthopedic screw to be properly positioned in a patient. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). The marker material can form all or a portion of the orthopedic screw and/or be coated on one or more portions of the orthopedic screw. The location of the marker material can be on one or multiple locations on the orthopedic screw. The size of the one or more regions including the marker material can be the same or different. The marker material can be spaced at defined distances from one another to form ruler-like markings on the orthopedic screw to facilitate in the positioning of the orthopedic screw in a body passageway. The marker material can be a rigid or flexible material. The marker material can be a biostable or biodegradable material.

In accordance with another and/or alternative aspect of the present disclosure, the orthopedic screw or one or more regions of the orthopedic screw can optionally be constructed by use of one or more microelectromechanical manufacturing (MEMS) techniques (e.g., micro-machining, laser micro-machining, micro-molding, etc.); however, other or additional manufacturing techniques can be used.

In accordance with another and/or alternative aspect of the present disclosure, a portion or all of the orthopedic screw that is formed of a metal alloy that includes at least 5 awt. % rhenium can optionally be formed by a near net process. In one non-limiting embodiment of the disclosure, there is provided a method of powder pressing materials and increasing the strength post-sintering by imparting additional cold work. In one non-limiting embodiment, a portion or all of the orthopedic screw that is formed of a metal alloy that includes at least 5 awt. % rhenium is formed of metal powder that is pressed and then sintered. Thereafter, the sintered part can optionally be again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered and re-pressed) meets the dimensional requirements of the final formed part. There is also provided a process of increasing the mechanical strength of a pressed metal part by repressing the post-sintered part to add additional cold work into the material, thereby increasing its mechanical strength. There is also provided a process of powder pressing to a near net or final part using metal powder. In one non-limiting embodiment, there is optionally provided a process of creating a metal part with pre-defined voids to create a trabecular or foam structure composed of mixing a metal and polymer powder, pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation, on the metal substrate, and the polymer residual has some desired biological affect (e.g., masking the metal from the body by encapsulation, promotion of cellular attachment and growth). The polymer and metal powders can be of varying sizes to create multiple voids—some large to create a pathway for cellular growth, and some small to create a ruff surface to promote cellular attachment. As can be appreciated, the polymer can be uniformly or non-uniformly dispersed with the metal powder. For example, if the final formed part is to have a uniform density and pore structure, the polymer material is uniformly dispersed with the metal powder prior to consolidating and pressing the polymer and metal powders together and then subsequently sintering together the metal powder to form the metal part or orthopedic screw. Alternatively, if the formed metal part or orthopedic screw is to have one or more channels, passageways, and/or voids on the outer surface and/or within the formed part or orthopedic screw, at least a portion of the polymer is not uniformly distributed with the metal powder, but instead is concentrated or forms all of the region that is to be the one or more channels, passageways, and/or voids on the outer surface and/or within the formed part or orthopedic screw such that when the polymer and metal powder is sintered, some or all of the polymer is degraded and removed from the part or orthopedic screw, thereby forming such one or more channels, passageways, and/or voids on the outer surface and/or within the formed part or orthopedic screw. As such, the use of the polymer in combination with metal powder and subsequent pressing and sintering can be used to form novel and customized shapes for the orthopedic screw or the near net form of the orthopedic screw. Generally, the polymer constitutes about 0.1-70 vol. % (and all values and ranges therebetween) of the consolidated and pressed material prior to the sintering step, typically the polymer constitutes about 1-60 vol. % of the consolidated and pressed material prior to the sintering step.

In accordance with another and/or alternative aspect of the present disclosure, a portion or all of the orthopedic screw that is formed of a metal alloy that includes at least 5 awt. % rhenium is initially formed into a blank, a rod, a tube, etc., and then finished into final form by one or more finishing processes. The metal alloy blank, rod, tube, etc., can be formed by various techniques such as, but not limited to, 1) melting the metal alloy and/or metals that form the metal alloy (e.g., vacuum arc melting, etc.) and then extruding and/or casting the metal alloy into a blank, rod, tube, etc., 2) melting the metal alloy and/or metals that form the metal alloy, forming a metal strip, and then rolling and welding the strip into a blank, rod, tube, etc., 3) consolidating the metal powder of the metal alloy and/or metal powder of metals that form the metal alloy into a blank, rod, tube, etc., or 4) 3-D printing the metal powder of the metal alloy and/or metal powder of metals that form the metal alloy into a blank, rod, tube, etc. When the metal alloy is formed into a blank, the shape and size of the blank is non-limiting. In one non-limiting process, the near net orthopedic screw, near net component of the orthopedic screw, blank, rod, tube, etc., can be formed from one or more ingots of metal or metal alloy. In one non-limiting process, an arc melting process (e.g., vacuum arc melting process, etc.) can be used to form the near net orthopedic screw, near net component of the orthopedic screw, blank, rod, tube, etc. In one non-limiting embodiment, the average particle size of the metal powders is less than about 230 mesh (e.g., less than 63 microns; 1-62 microns and all values and ranges therebetween). In another and/or alternative non-limiting embodiment, the average particle size of the metal powders is about 2-62 microns, and more particularly about 5-49.9 microns. In another and/or alternative non-limiting embodiment, the average particle size of the metal powders is about 10-40 microns. In another and/or alternative non-limiting embodiment, the average density of the metal powders is greater than 5 g/cm³ (e.g., 5.001 g/cm³ to 19.3 g/cm³ and all values and ranges therebetween). In another and/or alternative non-limiting embodiment, 10-100 vol. % (and all values and ranges therebetween) of the metal powder is spherical shaped. The purity of the metal powders should be selected so that the metal powders contain very low levels of carbon, oxygen, and nitrogen. Typically, the carbon content of the metal powder used to form the metal alloy is less than about 100 ppm, the oxygen content is less than about 50 ppm, and the nitrogen content is less than about 20 ppm. Typically, metal powder used to form the metal alloy has a purity grade of at least 99.9 and more typically at least about 99.95.

In accordance with another and/or alternative aspect of the present disclosure, when the metal powder is consolidated to form the metal alloy into a blank, rod, tube, etc., the metal powder is pressed together to form a solid solution of the metal alloy into a near net orthopedic screw, near net component of the orthopedic screw, blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.), and/or under a vacuum; however, this is not required. The average density of the near net orthopedic screw, near net component of the orthopedic screw, blank, rod, tube, etc., that is achieved by pressing together the metal powders is about 80-95% (and all values and ranges therebetween) of the final average density of the near net orthopedic screw, near net component of the orthopedic screw, blank, rod, tube, etc., or about 70-96% (and all values and ranges therebetween) the minimum theoretical density of the metal alloy. Pressing pressures of at least about 300 MPa are generally used. Generally, the pressing pressure is about 400-700 MPa; however, other pressures can be used. After the metal powders are pressed together, the pressed metal powders are sintered to partially or fully fuse the metal powders together to form the near net orthopedic screw, near net component of the orthopedic screw, blank, rod, tube, etc. The sintering of the consolidated metal powder can be performed in an oxygen-reducing atmosphere (e.g., helium, argon, hydrogen, argon and hydrogen mixture, etc.), and/or under a vacuum; however, this is not required. At the high sintering temperatures, a high hydrogen atmosphere will reduce both the amount of carbon and oxygen in the formed near net orthopedic screw, near net component of the orthopedic screw, blank, rod, tube, etc. The sintered metal powder generally has an as-sintered average density of about 90-99% the minimum theoretical density of the metal alloy.

In accordance with another and/or alternative aspect of the present disclosure, when the metal powder is used to 3D print the orthopedic screw, component of the orthopedic screw, blank, rod, tube, etc., the average particle size of the metal powder is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween).

In accordance with another and/or alternative aspect of the present disclosure, the near net orthopedic screw, near net component of the orthopedic screw, blank, rod, tube, etc., can optionally be cleaned and/or polished after the near net orthopedic screw, near net component of the orthopedic screw, blank, rod, tube, etc., has been formed; however, this is not required.

In accordance with another and/or alternative aspect of the present disclosure, the near net orthopedic screw, near net component of the orthopedic screw, blank, rod, tube, etc., can be resized to the desired dimension of the orthopedic screw. In one non-limiting embodiment, the cross-sectional area or diameter of the near net orthopedic screw, near net component of the orthopedic screw, blank, rod, tube, etc., is reduced to a final near net orthopedic screw, near net component of the orthopedic screw, blank, rod, tube, etc. in a single step or by a series of steps. The reduction of the outer cross-sectional area or diameter of the near net orthopedic screw, near net component of the orthopedic screw, blank, rod, tube, etc., may be obtained by centerless grinding, turning, electropolishing, drawing process, grinding, laser cutting, shaving, polishing, EDM cutting, etc. The outer cross-sectional area or diameter size of the near net orthopedic screw, near net component of the orthopedic screw, blank, rod, tube, etc., can be reduced by the use of one or more drawing processes; however, this is not required. During the drawing process, care should be taken to not form micro-cracks in the near net orthopedic screw, near net component of the orthopedic screw, blank, rod, tube, etc., during the reduction of the near net orthopedic screw, near net component of the orthopedic screw, blank, rod, tube, etc., outer cross-sectional area or diameter.

In accordance with another and/or alternative aspect of the present disclosure, the orthopedic screw can be partially or fully formed by one or more manufacturing processes. These manufacturing processes can include, but are not limited to, laser cutting, etching, annealing, nitriding, drawing, pilgering, electroplating, electro-polishing, machining, plasma coating, 3D printed coatings, 3D printing, chemical vapor deposition, chemical polishing, cleaning, pickling, ion beam deposition or implantation, sputter coating, vacuum deposition, injection molding, etc.

The use of the metal alloy that includes at least 5 awt. % rhenium to form all or a portion of the orthopedic screw can result in several advantages over orthopedic screw having a similar size and/or shape and formed from other materials. These advantages include, but are not limited to:
The metal alloy that includes at least 5 awt. % rhenium has increased strength and/or hardness as compared with stainless steel or chromium-cobalt alloys or titanium alloys, thus a lesser quantity of metal alloy can be used in the orthopedic screw to achieve similar strengths compared to orthopedic screws formed of different metals. As such, the resulting orthopedic screw can be made smaller and less bulky by use of the metal alloy without sacrificing the strength and durability of the orthopedic screw. The orthopedic screw can also have a smaller profile, thus can be inserted into smaller bones and/or result in smaller size tap holes in the bone, thereby reducing potential injury to the bone.
The metal alloy that includes at least 5 awt. % rhenium has improved stress-strain properties, bendability properties, elongation properties, and/or flexibility properties of the orthopedic screw compared with stainless steel or chromium-cobalt alloys, thus resulting in an increased life for the orthopedic screw. For instance, the orthopedic screw can be used in regions that subject the orthopedic screw to repeated bending. Due to the improved physical properties of the orthopedic screw from the metal alloy, the orthopedic screw has improved resistance to fracturing in such frequent bending environments.
The metal alloy that includes at least 5 awt. % rhenium can have improved fatigue ductility when subjected to cold-working as compared to the cold-working of stainless steel, chromium-cobalt alloys, or titanium alloys.
The metal alloy that includes at least 5 awt. % rhenium can have improved durability compared to stainless steel, chromium-cobalt alloys, or titanium alloys.
The metal alloy that includes at least 5 awt. % rhenium can have improved hydrophilicity compared to stainless steel, chromium-cobalt alloys, or titanium alloys.
The metal alloy that includes at least 5 awt. % rhenium can have reduced ion release after implantation in a patient as compared to stainless steel, chromium-cobalt alloys, or titanium alloys.
The metal alloy that includes at least 5 awt. % rhenium can be less of an irritant to the body than stainless steel, cobalt-chromium alloy, or titanium alloys, thus can result in reduced inflammation, faster healing, and increased success rates when using the orthopedic screw.

In another and/or alternative non-limiting aspect of the disclosure, the orthopedic screw includes a head portion and a body portion. The head portion generally has a maximum cross-sectional area that is greater than a maximum cross-sectional area of the body portion. The head portion may or may not include threads. Likewise, the body portion may or may not include threads. The threads (when used) are generally spiral shaped. For example, the body portion can be fully threaded, partially threaded, comprise a spiral or helical blade, and/or may comprise one or more tacks, deployable talons, expanding elements, or so forth. As can be appreciated, the body of the screw can be a peg or pin shape. The threads on the head portion and/or body portion of the screw (when used) are non-limiting (e.g., right-hand threads, left-band threads, taper threads, “V” shape threads, metric threads, British threads, seller threads, square threads, acme threads, buttress threads, knuckle threads, worm threads, single and multi-threads). The end region of the body portion can optionally include a self-tapping or self-drilling tip; however, this is not required. The shape of the head portion is non-limiting. In one non-limiting embodiment, the cross-sectional shape of top region of the head portion is generally circular shaped; however, other shapes can be used. The head portion can optionally include a cavity to facilitate in inserting the orthopedic screw into a bone and securing the bone plate to the bone. The configuration of the cavity (when used) is non-limiting. Generally, the cavity is specially shaped to receive a tool to rotate and/or otherwise cause the orthopedic screw to be inserted into a bone. Non-limiting shapes that can be used in the cavity include one or more dimples, ridges, bumps, textured areas, star shaped, polygonal shaped, or any other surface or shape. As can be appreciated, the cavity can include a threaded inner surface and a circular-cross-sectional shape.

In another and/or alternative non-limiting aspect of the disclosure, the orthopedic screw includes a head portion and a body portion, the head portion generally has a maximum cross-sectional area that is greater than a maximum cross-sectional area of the body portion, 60-100% (and all values and ranges therebetween) of the longitudinal length of the head portion is absent threads, 60-100% (and all values and ranges therebetween) of the longitudinal length of the body portion includes spirally-shaped threads, the end region of the body portion optionally includes a self-tapping or self-drilling tip, the cross-sectional shape of the top region of the head portion is generally circular shaped along 60-100% (and all values and ranges therebetween) of the longitudinal length of the head portion, the cross-sectional shape of the bottom region of the head portion is generally circular shaped along 60-100% (and all values and ranges therebetween) of the longitudinal length of the head portion, and the head portion can optionally include a cavity to facilitate in inserting the orthopedic screw into a bone and securing the bone plate to the bone.

In another and/or alternative non-limiting aspect of the disclosure, the orthopedic screw includes a head portion and a body portion, the head portion generally has a maximum cross-sectional area that is greater than a maximum cross-sectional area of the body portion, 60-100% (and all values and ranges therebetween) of the longitudinal length of the head portion includes threads, 60-100% (and all values and ranges therebetween) of the longitudinal length of the body portion includes spirally-shaped threads, the end region of the body portion optionally includes a self-tapping or self-drilling tip, the cross-sectional shape of the top region of the head portion is generally circular shaped along 60-100% (and all values and ranges therebetween) of the longitudinal length of the head portion, the cross-sectional shape of the bottom region of the head portion is generally circular shaped along 60-100% (and all values and ranges therebetween) of the longitudinal length of the head portion, and the head portion can optionally include a cavity to facilitate in inserting the orthopedic screw into a bone and securing the bone plate to the bone.

In another and/or alternative non-limiting aspect of the disclosure, the head portion of the orthopedic screw has a tapered profile that includes threading along 60-100% (and all values and ranges therebetween) of the central axis of the tapered portion of the head portion. The tapered portion of the head portion is generally 20-100% (and all values and ranges therebetween) of the longitudinal length of the head portion. The angle of taper is generally at least 1° (e.g., 1-25° and all values and ranges therebetween). As such, the cross-sectional area and/or diameter of the top region of the head portion that includes threading on the tapered region is greater than the cross-sectional area and/or diameter of the bottom region of the head portion that includes threading on the tapered region. In one non-limiting arrangement, the angle of taper of the head portion that includes threading is constant along 60-100% (and all values and ranges therebetween) of the taper. In another non-limiting arrangement, the threading on the head portion is spirally shaped and has a) a constant width along 60-100% (and all values and ranges therebetween) the length of the threading, b) a constant shape along 60-100% (and all values and ranges therebetween) the length of the threading, c) a constant lead along 60-100% (and all values and ranges therebetween) the length of the threading, and/or a constant pitch along 60-100% (and all values and ranges therebetween) the length of the threading. The use of the tapered region on the head portion of the orthopedic screw can optionally facilitate in enabling the orthopedic screw to be oriented in the screw opening at a desired angle relative to the central axis of the screw opening.

In another and/or alternative non-limiting aspect of the disclosure, the orthopedic screw optionally includes a non-threaded transition region that is positioned between the threading on the upper portion of head portion and the threading on the top portion of the body portion. The non-threaded transition region of the head portion (when used) is generally 5-80% (and all values and ranges therebetween) of the longitudinal length of the head portion. In one non-limiting arrangement, the minimum cross-sectional area of the non-threaded transition region is less than the maximum cross-sectional area of the head portion and/or the body portion. The use of the non-threaded transition region on the head portion of the orthopedic screw can optionally facilitate in enabling the orthopedic screw to be oriented in the screw opening at a desired angle relative to the central axis of the screw opening.

In another and/or alternative non-limiting aspect of the disclosure, the threading on the head portion of the orthopedic screw generally has the same or similar lead and pitch as the threading in the screw opening in the bone plate.

In another and/or alternative non-limiting aspect of the disclosure, the threading on the body portion of the orthopedic screw includes threading that has a different lead and pitch than the threading on the head portion of the orthopedic screw.

In another and/or alternative non-limiting aspect of the disclosure, the threading on the body portion of the orthopedic screw has a maximum cross-sectional area that is less than the minimum cross-sectional area of the screw opening so the body portion of the orthopedic screw can be passed through the screw opening without having to be threaded through the threads on the screw opening.

In one non-limiting object of the present disclosure, there is the provision of an orthopedic screw that is at least partially formed of a metal alloy that includes at least 5 awt. % rhenium.

In another and/or alternative non-limiting object of the present disclosure, there is the provision of an orthopedic screw that includes a head portion and a body portion that is connected to the bottom of the head portion and extends downwardly from the head portion; the head portion includes head threading on an outer surface of the head portion; the body portion includes body threading on an outer surface of the body portion; one or more of a) a pitch of the body threading is different from a pitch of the head threading, b) a thread length of the body threading is different from a thread length of the head threading, c) a thread angle of the body threading is different from a thread angle of the head threading, d) a root depth of the body threading is different from a root depth of the head threading, and/or e) a lead of the body threading is different from a lead of the head threading.

In another and/or alternative non-limiting object of the present disclosure, there is the provision of an orthopedic screw that includes a tapered portion that forms a tapered profile of the head portion along a central axis of said the portion; the head threading is at least partially located on the tapered portion; the tapered portion constitutes 20-100% of a longitudinal length of the head portion.

In another and/or alternative non-limiting object of the present disclosure, there is the provision of an orthopedic screw that includes a tapered portion and wherein an angle of taper of the tapered portion is constant along 60-100% of the tapered portion.

In another and/or alternative non-limiting object of the present disclosure, there is the provision of an orthopedic screw that includes a tapered portion and wherein the head threading on the tapered portion has a tapered profile.

In another and/or alternative non-limiting object of the present disclosure, there is the provision of an orthopedic screw wherein said body portion includes a tapered transition region that terminates at said head portion; said transition region absent threading.

In another and/or alternative non-limiting object of the present disclosure, there is the provision of an orthopedic screw wherein a bottom portion of said body portion includes a cut face; said cut face intersects a portion of said body threading thereby forming a break in said body threading.

In another and/or alternative non-limiting object of the present disclosure, there is the provision of an orthopedic screw wherein a top surface of said head portion has a non-circular profile.

In another and/or alternative non-limiting object of the present disclosure, there is the provision of an orthopedic screw wherein a top surface of said head portion has a non-circular profile, and wherein said top surface of said head portion includes a plurality of curvilinear regions having different radii.

In another and/or alternative non-limiting object of the present disclosure, there is the provision of an orthopedic screw that is partially or fully formed of an improved metal alloy, and wherein the orthopedic screw has a cross-sectional area of the root that is at least 10% less that a cross-sectional area of a similar shaped screw formed of stainless steel, cobalt chromium alloy, or titanium alloy, and wherein the orthopedic screw is partially or fully formed of a metal alloy that includes at least 5 awt. % rhenium and has i) the same or greater tensile strength as compared to a similar shaped an orthopedic screw formed of stainless steel, cobalt chromium alloy, or titanium alloy, ii) the same or greater ductility as compared to a similar shaped an orthopedic screw formed of stainless steel, cobalt chromium alloy, or titanium alloy, iii) a reduced corrosion rate as compared a similar shaped an orthopedic screw formed of stainless steel, cobalt chromium alloy, or titanium alloy, iv) a reduced ion release rate as compared to a similar shaped an orthopedic screw formed of stainless steel, cobalt chromium alloy, or titanium alloy, v) the same or greater hardness as compared to a similar shaped an orthopedic screw formed of stainless steel, cobalt chromium alloy, or titanium alloy, and/or vi) the same or greater yield strength as compared to a similar shaped an orthopedic screw formed of stainless steel, cobalt chromium alloy, or titanium alloy.

In another and/or alternative non-limiting object of the present disclosure, there is the provision of an orthopedic screw that is partially or fully formed of a metal alloy that includes at least 5 awt. % rhenium, and wherein the thread on the head portion of the orthopedic screw has a cross-sectional area that is at least 10% less that a cross-sectional area of a thread on a head portion of a similar shaped orthopedic screw formed of stainless steel, cobalt chromium alloy, or titanium alloy, and wherein the orthopedic screw that is partially or fully formed of the metal alloy that includes at least 5 awt. % rhenium has i) the same or greater tensile strength as compared to a similar shaped an orthopedic screw formed of stainless steel, cobalt chromium alloy, or titanium alloy, ii) the same or greater ductility as compared to a similar shaped an orthopedic screw formed of stainless steel, cobalt chromium alloy, or titanium alloy, iii) a reduced corrosion rate as compared a similar shaped an orthopedic screw formed of stainless steel, cobalt chromium alloy, or titanium alloy, iv) a reduced ion release rate as compared to a similar shaped an orthopedic screw formed of stainless steel, cobalt chromium alloy, or titanium alloy, v) the same or greater hardness as compared to a similar shaped an orthopedic screw formed of stainless steel, cobalt chromium alloy, or titanium alloy, and/or vi) the same or greater yield strength as compared to a similar shaped an orthopedic screw formed of stainless steel, cobalt chromium alloy, or titanium alloy.

In another and/or alternative non-limiting object of the present disclosure, there is the provision of an orthopedic screw that is partially or fully formed of a metal alloy that includes at least 5 awt. % rhenium, and wherein the thread on the body portion of the screw has a cross-sectional area that is at least 10% less that a cross-sectional area a thread on the body portion of a similar shaped screw formed of stainless steel, cobalt chromium alloy or titanium alloy, and wherein the orthopedic screw that is partially or fully formed of the metal alloy that includes at least 5 awt. % rhenium has i) the same or greater tensile strength as compared to a similar shaped an orthopedic screw formed of stainless steel, cobalt chromium alloy, or titanium alloy, ii) the same or greater ductility as compared to a similar shaped an orthopedic screw formed of stainless steel, cobalt chromium alloy, or titanium alloy, iii) a reduced corrosion rate as compared a similar shaped an orthopedic screw formed of stainless steel, cobalt chromium alloy, or titanium alloy, iv) a reduced ion release rate as compared to a similar shaped an orthopedic screw formed of stainless steel, cobalt chromium alloy, or titanium alloy, v) the same or greater hardness as compared to a similar shaped an orthopedic screw formed of stainless steel, cobalt chromium alloy, or titanium alloy, and/or vi) the same or greater yield strength as compared to a similar shaped an orthopedic screw formed of stainless steel, cobalt chromium alloy, or titanium alloy.

These and other objects and advantages will become apparent from the discussion of the distinction between the disclosure and the prior art and when considering the non-limiting embodiment illustrated in the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIGS. 1-2 illustrate a prior art Ti6Al4V bone screw.

FIGS. 3 and 4 are side views of a non-limiting orthopedic screw in accordance with the present disclosure.

FIG. 5 is a cross-sectional view of the bone plate along line 4-4 of FIGS. 3 and 4.

FIG. 6 is a top plan view of the bone screw of FIG. 3.

FIG. 7 is an enlarged end portion of a bone plate that includes an upper portion of a non-limiting orthopedic screw in accordance with the present disclosure that is positioned in the screw opening of the bone plate.

FIG. 8 is a top plan view of FIG. 7.

FIG. 9 is a cross-sectional view of the bone plate along line 9-9 of FIG. 7.

FIG. 10 is top view of a bone plate that is secured to a bone structure by non-limiting orthopedic screws in accordance with the present disclosure.

FIG. 11 is a side elevation view of another non-limiting orthopedic screw in accordance with the present disclosure.

FIG. 12 is a top view of the orthopedic screw of FIG. 11.

FIG. 13 is a bottom view of the orthopedic screw of FIG. 11.

FIG. 14 is a front view of the orthopedic screw of FIG. 11.

FIG. 15 is an enlarged side view of a top portion of the orthopedic screw of FIG. 11.

FIG. 16 is a cross-sectional view of the head of the orthopedic screw along line 16-16 of FIG. 15.

FIG. 17 is a side view of the orthopedic screw of FIG. 11.

FIG. 18 is a cross-sectional view of the head of the orthopedic screw along line 18-18 of FIG. 17.

FIG. 19 is a side view of an enlarged head portion of the orthopedic screw of FIG. 11.

FIG. 20 is a cross-sectional view of the head portion of the orthopedic screw of FIG. 19.

FIGS. 21-23 are front views of the orthopedic screw of FIG. 11 prior to the threads being cut in the bottom portion of the orthopedic screw.

FIG. 24 is an enlarged cross-sectional view of the bottom portion of the orthopedic screw of FIG. 11.

FIG. 25 is an enlarged sectional portion of the side of the middle region of the orthopedic screw of FIG. 11.

FIG. 26 is an enlarged sectional portion of the side of the top portion of the orthopedic screw of FIG. 11.

### DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

All ranges includes in the present disclosure include the end points of the range as well as all values and ranges therebetween of such ranges unless indicated otherwise.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

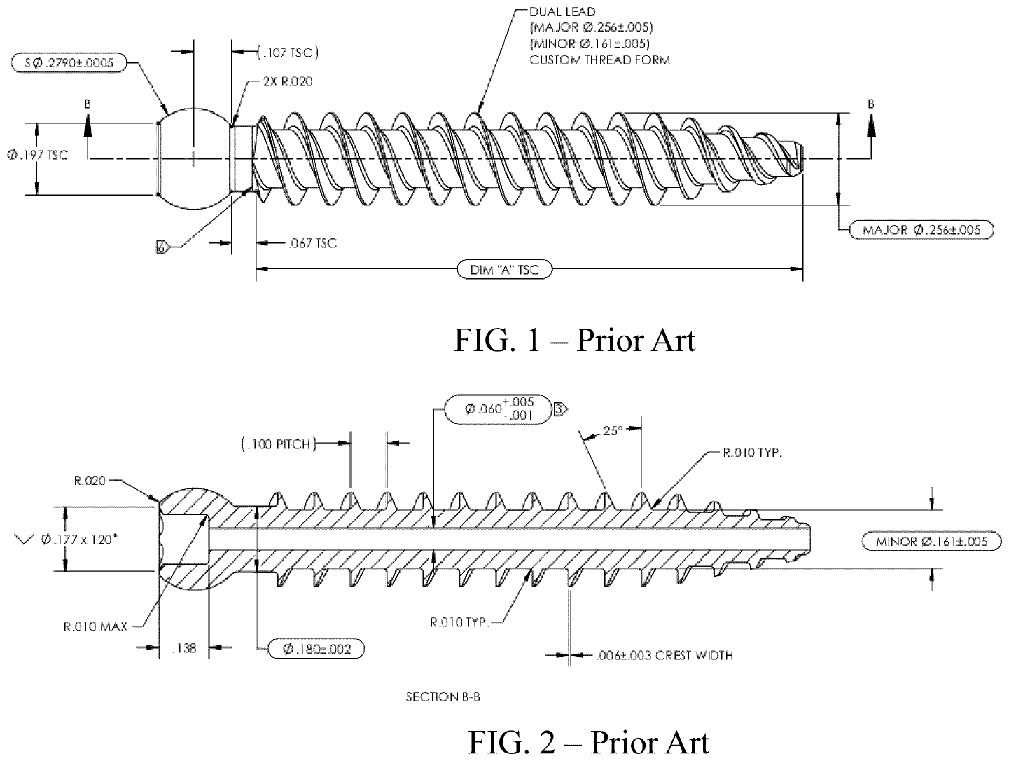

Referring now to FIG. 1-2, there is illustrated a prior art bone screw that is formed of a titanium alloy (e.g., Ti6Al4V). Titanium alloy screws are a common type of bone screw used to secure plate plates to various type of bones. The bone screw has a top portion and a bottom portion. Typically the top portion is absent any type of threading. The top end of the bone screw generally includes a cavity (e.g., non-circular cavity, threaded cavity, etc.) configured to accept a tool that is used to insert the bone screw into a bone to secure the bone plate to the bone. The top end is generally absent threading on the outer surface of the top end. The top end is shaped as a spherical segment or a spherical slice wherein the top end has a shape of a sphere with the top and bottom portions of the sphere removed. The bottom portion of the bone screw generally includes threading. The threading is illustrated as beginning as a location that is spaced from the top portion. The end portion of the bottom portion is generally tapered. The bone screw may or may not be cannulated (e.g., a cavity along the longitudinal axis of the screw. FIGS. 1-2 provide prior art dimensions of the major and minor diameters of the threading of the bone screw.

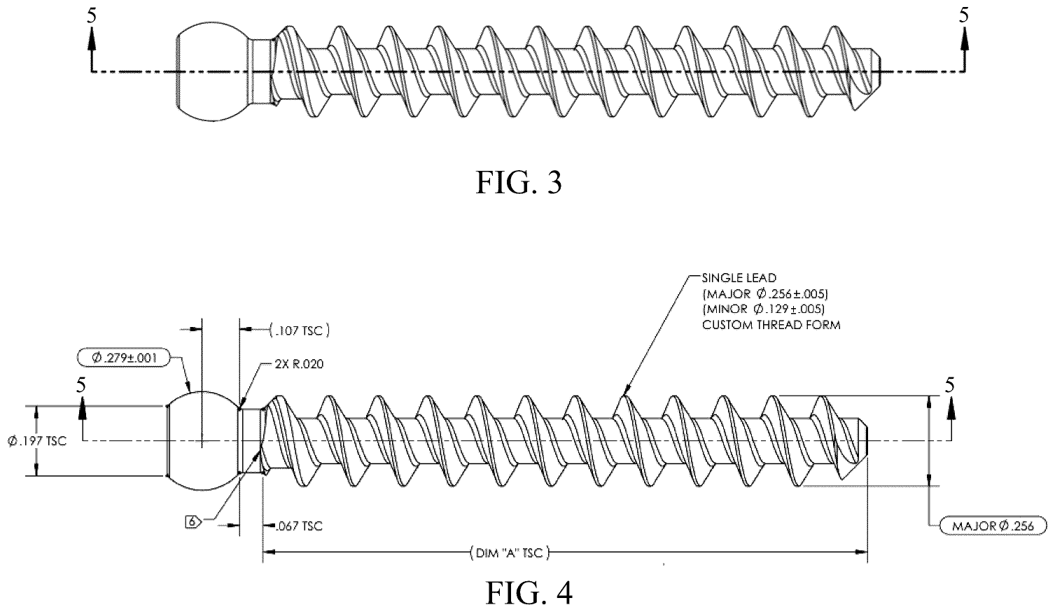

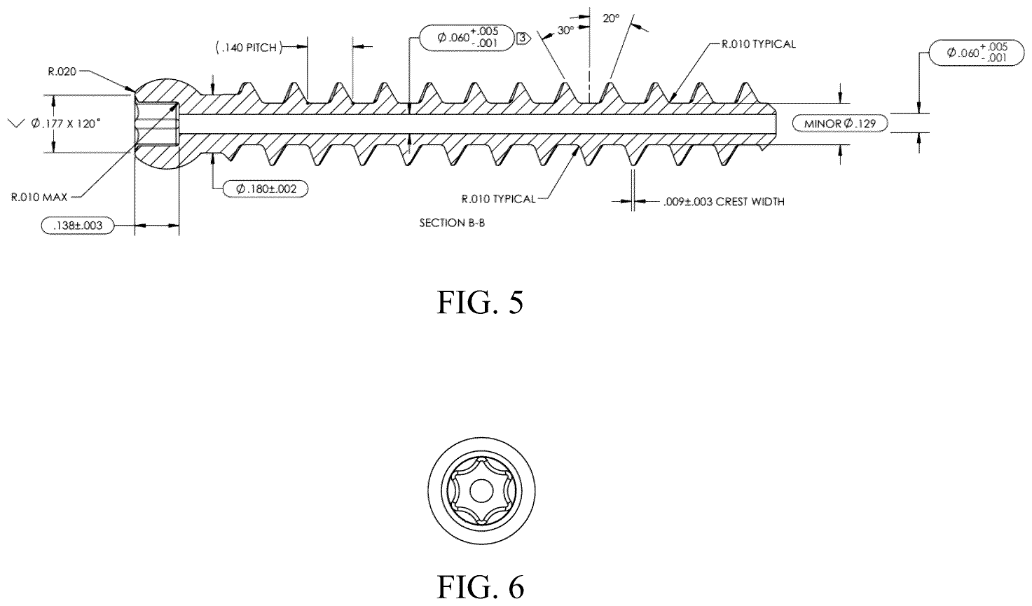

Referring now to FIGS. 3-6, there is illustrated a similar shaped orthopedic screw as illustrated in FIGS. 1-2; however, the orthopedic screw in accordance with the present disclosure is formed of a rhenium-containing metal alloy, and has a different longitudinal profile. The bone screw illustrated in FIGS. 3-6 has the same or greater strength as the titanium alloy bone screw illustrated in FIGS. 1-2, but the minor diameter of the threading of the orthopedic screw illustrated in FIGS. 3-6 is at least 5% (e.g., 5%-30% and all values and ranges therebetween) smaller than the minor diameter of the threading of the titanium alloy bone screw illustrated in FIGS. 1-2. The major diameter, the longitudinal length, the general shape of the threading, and shape of the head of the bone screw of FIGS. 1-2 and the orthopedic screw of FIGS. 3-6 are illustrated as being the same or similar. The ability to form an orthopedic screw in accordance with the present disclosure with a smaller minor diameter (e.g., 5-30% smaller minor diameter and all values and ranges therebetween) without having to sacrifice the strength of the orthopedic screw as compared to a standard titanium alloy screw as illustrated in FIG. 1-2 results in 1) less material being required for forming the orthopedic screw, 2) potentially less weight of the orthopedic screw due to less material used to form the orthopedic screw, and/or 3) a stronger connection between the orthopedic screw and bone due to the differential between the major and minor diameters of the thread. As can also be appreciated, the orthopedic screw in accordance with the present disclosure can have different shapes, threading shapes, and profiles, etc., from the shape and threading profile illustrated in FIGS. 3-6. As illustrated in FIGS. 3-6, the orthopedic screw does not have a tapered section along the threaded portion of the orthopedic screw; which configuration is different from the prior art orthopedic screw illustrated in FIGS. 1-2. It will be appreciated that the dimensions illustrated in FIGS. 4-5 are non-limiting dimensions of one non-limiting configuration of the orthopedic screw of FIGS. 3-6, thus other dimensions of the orthopedic screw can be used in accordance with the present disclosure.

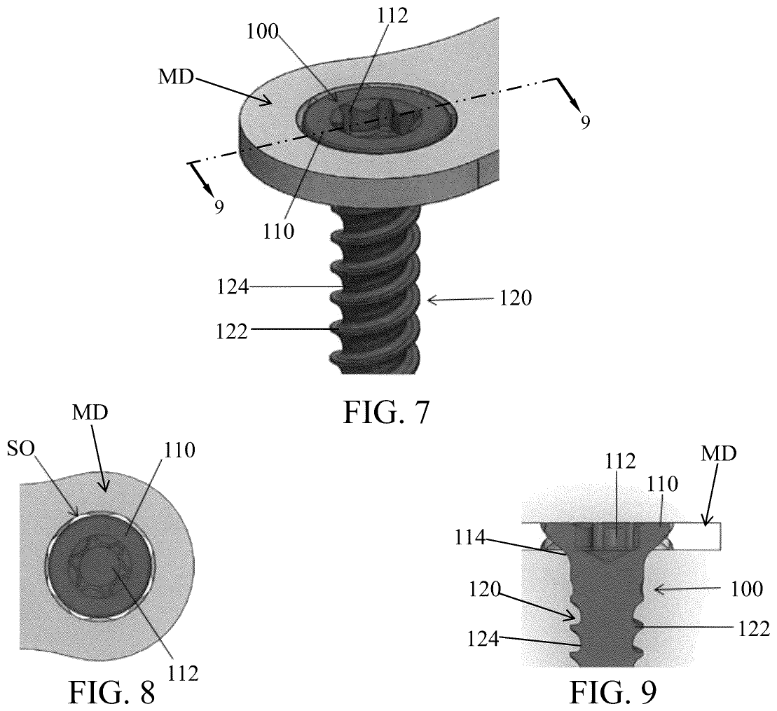

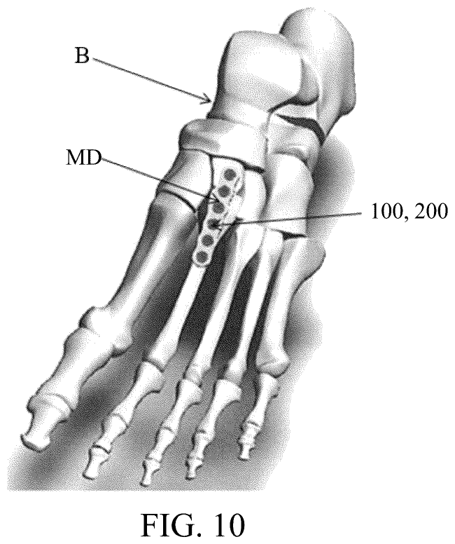

Referring now to FIGS. 7-10, there is illustrated an orthopedic screw 100 that can be used as a connection arrangement for use in various orthopedic applications. Orthopedic screw 100 can be used on various type of medical devices MD to at least partially secure medical device MD to a bone B such as, but not limited to, a radius, an ulna, spinal bones, maxillofacial bones, a fibula, metatarsal bones, calcaneus bones, ankle bones, femur, distal tibia, proximal tibia, proximal humerus, distal humerus, a clavicle, bones of the foot, bones of the hand, etc. As can be appreciated, orthopedic screw 100 in accordance with the present disclosure can have other applications. One or more or all portions of orthopedic screw 100 can optionally be formed by a cutting process, stamping process, 3D printer, molding process, sintering process, near net process, etc.

Orthopedic screw 100 can be partially or fully formed of a metal alloy that includes at least 5 awt. % rhenium. Orthopedic screw 100 can be configured to penetrate and affix a medical device MD to a bone B.

Orthopedic screw 100 includes a head portion 110 and a body portion 120. Body portion 120 includes threads 122 and a screw root 124. Body portion 120 can be fully or partially threaded. The type of threads (e.g., right-hand threads, left-hand threads, taper threads, “V” shape threads, metric threads, British threads, seller threads, square threads, acme threads, buttress threads, knuckle threads, worm threads, single and multi-threads), the pitch of the threads, the thread angle, the cross-sectional area or diameter of the root, the thread length, etc. is non-limiting.

The end of body portion 120 (not shown) can optionally include a self-tapping or self-drilling tip.

The maximum cross-sectional size of head portion 110 of the orthopedic screw is greater than the maximum cross-sectional size of body portion 120. The cross-sectional shape of the top region of head portion 120 is generally circular shaped. Head portion 120 can optionally include a cavity 112 to facilitate in inserting the orthopedic screw into a bone and securing medical device MD to the bone B. The configuration of cavity 112 is non-limiting. Generally, cavity 112 is specially shaped (e.g., hexagon shaped, six point star shaped, etc.) to receive a tool to rotate and/or otherwise cause orthopedic screw 100 to be inserted into a bone. Non-limiting shapes that can be used in the cavity include one or more dimples, ridges, bumps, textured areas, or any other surface. As illustrated in FIG. 2, cavity 112 as a star-shape. The depth of cavity 112 is generally the same as or less than the longitudinal length of head portion 110 as illustrated in FIG. 9.

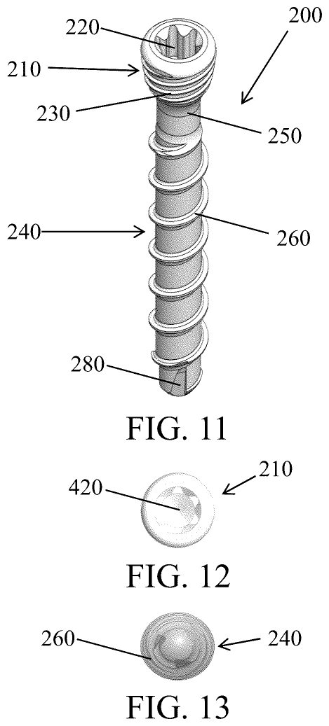

Head portion 110 may or may not include threading on the outer surface. As illustrated in FIG. 3, head portion 110 is absent threading. As illustrated in FIG. 11, the head portion includes threading on the outer surface.

Head portion 110 can optionally include a tapered region 114 that transitions to body portion 120. The start of thread 122 are illustrated to begin at location that is spaced from the head portion 110.

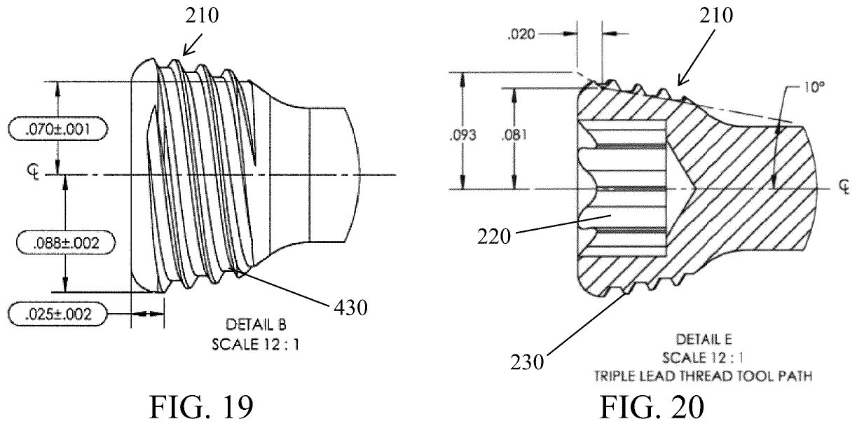

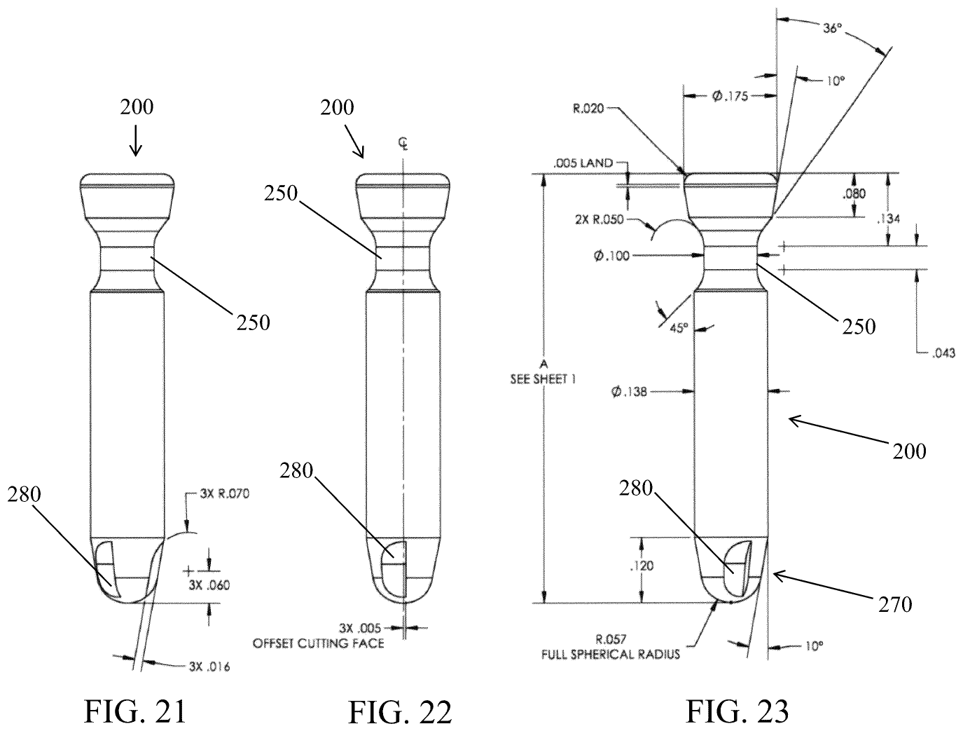

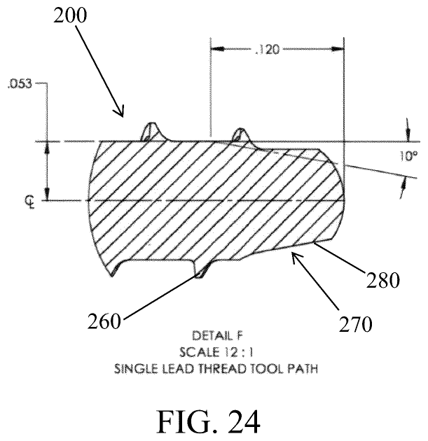

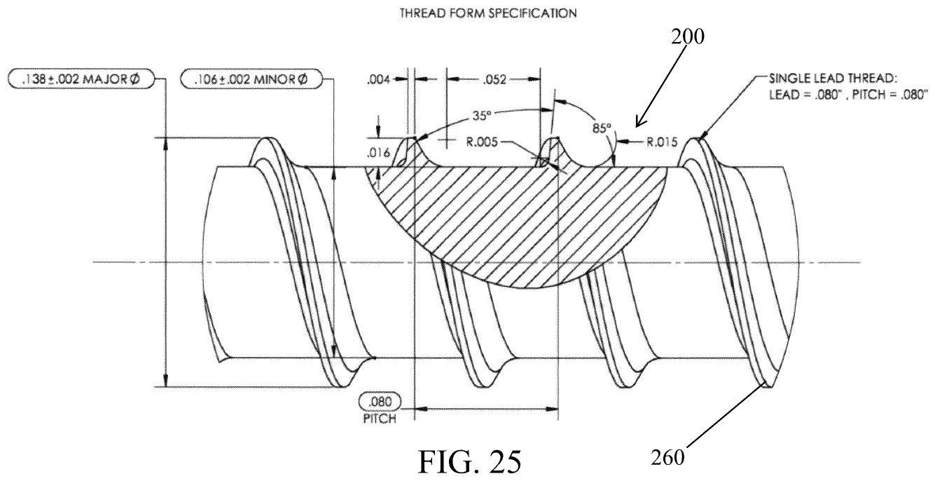

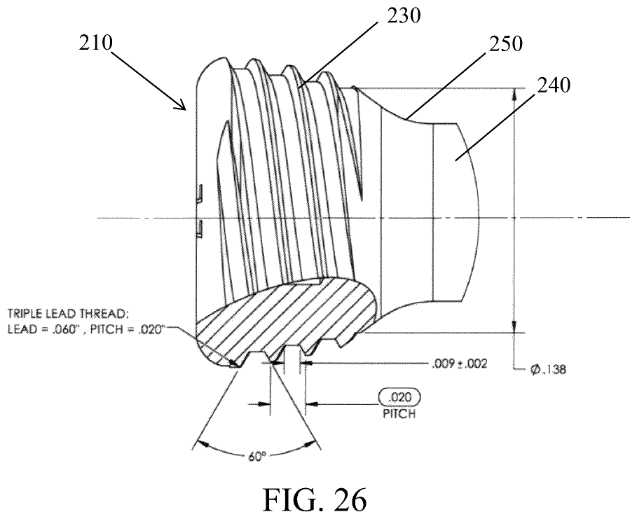

Referring now to FIGS. 11-20, another non-limiting orthopedic screw 200 is illustrated, Orthopedic screw 200 includes a bead portion 210 and a body portion 240 that is connected to the bottom of head portion 210 and extends downwardly from head portion 210.

Head portion 210 includes a top opening 220 and head threading 230 on the outer surface of head portion 210.

Top opening 220 generally includes a non-circular cross-sectional shape or a threaded inner surface. As illustrated in FIG. 12, top opening 220 has a hexagonal cross-sectional shape or a hexagon star shape along at least a portion of the central axis of the opening 220. As can be appreciated, other non-circular cross-sectional shapes can be used (e.g., triangular, square, polygonal, oval, etc.), As illustrated in FIG. 11 the depth of top opening 220 along the central axis of top opening 220 is generally at least 30% (e.g., 30-100% and all values and ranges therebetween) the depth of head portion 210 along the central axis of head portion 210. Top opening 220 is generally spaced from the other perimeter of head portion 210.

Referring now to FIG. 15, head portion 210 has a tapered profile along the central axis of head portion 210. The angle of taper α is generally at least 1° (e.g., 1-25° and all values and ranges therebetween). In one non-limiting configuration, the angle of taper α of head portion 210 is 10-20°. As such, the cross-sectional area and/or diameter of the top of the head portion 210 is greater than the cross-sectional area and/or diameter of the bottom of head portion 210. As illustrated in FIG. 15, the cross-sectional area and/or diameter of head threading 230 from the top to the bottom of head 210 portion also progressively reduces due to the taper of the head portion.

The tapered profile includes head threading 230 along 60-100% (and all values and ranges therebetween) of the central axis of the tapered portion of the head portion. The tapered portion of the head portion is generally 20-100% (and all values and ranges therebetween) of the longitudinal length of the head portion. The angle of taper of the head portion can be constant along 60-100% (and all values and ranges therebetween) of the taper, Head threading 230 on the head portion is spirally shape and has a) a constant width along 60-100% (and all values and ranges therebetween) the length of the threading, b) a constant shape along 60-100% (and all values and ranges therebetween) the length of the threading, c) a constant lead along 60-100% (and all values and ranges therebetween) the length of the threading, and/or a constant pitch along 60-100% (and all values and ranges therebetween) the length of the threading. The threading 230 is illustrated as terminating at or before the transition portion 250.

The use of the tapered region on the head portion of the orthopedic screw can optionally facilitate in enabling the orthopedic screw to be oriented in orthopedic screw opening SO at a desired angle relative to the central axis of a medical device MD. In one non-limiting embodiment, the bottom portion of the tapered region on the head portion of the orthopedic screw has a cross-sectional diameter and/or cross-section area that is at least 5% less (e.g., 5-25% less and all values and ranges therebetween) than the cross-sectional diameter and/or cross-section area of the top of orthopedic screw opening SO. In another non-limiting embodiment, the top portion of the tapered region on the head portion of the orthopedic screw has a cross-sectional diameter and/or cross-section area that is greater than 95% (e.g., 95-100% all values and ranges therebetween) of the cross-sectional diameter and/or cross-section area of the top of orthopedic screw opening SO.

The lead of head threading 230 is generally greater (e.g., 5-1000% greater and all values and ranges therebetween) than the pitch of head threading 230; however, this is not required. In one non-limiting embodiment, the lead of head threading 230 is about 0.04-0.1 inches (and all values and ranges therebetween), and the pitch of head threading 230 is about 0.01-0.05 inches (and all values and ranges therebetween). In one non-limiting specific configuration, the lead is about 0.06 inches and the pitch is about 0.02 inches. Head threading 230 is illustrated as encircling head portion 210 at least once (e.g., 1-6 encirclements of head portion 210 and all values and ranges therebetween).

In one non-limiting configuration, the angle of head threading 230 is about 60° (e.g., 60°+20° and all values and ranges therebetween); however, other angles can be used.

Head portion 210 generally has a maximum cross-sectional area that is greater than a maximum cross-sectional area of body portion 240. The shape of head portion 210 is non-limiting. In one non-limiting configuration, the cross-sectional shape of the top region of head portion 210 is generally circular shaped.

As illustrated in FIG. 11, orthopedic screw 200 can optionally include a transition portion 250 that is located from the bottom portion of head portion 210 to the top of body portion 240; however, this is not required. Transition portion 250 (when used) can include one or more curved regions; however, this is not required. The non-threaded transition region of the head portion (when used) is generally 5-80% (and all values and ranges therebetween) of the longitudinal length of the head portion. In one non-limiting configuration, the minimum cross-sectional area of the non-threaded transition region is less than the maximum cross-sectional area of the bead portion and/or the body portion, and the non-threaded transition region has a generally circular cross-sectional shape along 60-100% (and all values and ranges therebetween) of the longitudinal length of the non-threaded transition region 250. The use of the non-threaded transition region on the head portion of the orthopedic screw optionally facilitates in enabling the orthopedic screw to be oriented in screw opening SO of a medical device MD at a desired angle.

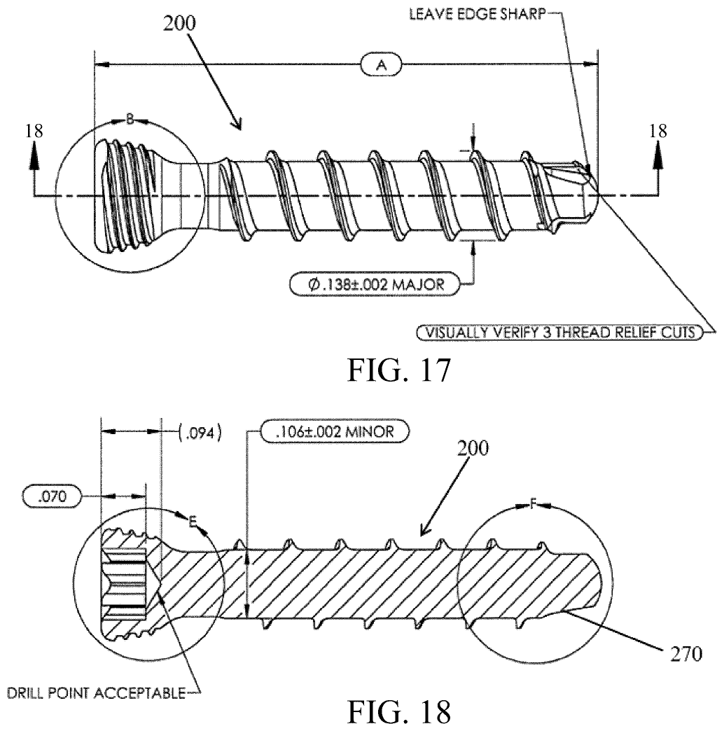

Referring now to FIGS. 11 and 17, body portion 240 of orthopedic screw 200 includes body threading 260 that is configured to be secured into a bone. Body threading 260 generally has a different lead and pitch than head threading 230 on the head portion 210 of orthopedic screw 200. In one non-limiting configuration, head threading 230 of orthopedic screw 200 has a lead of 0.04-0.08 inches (and all values and ranges therebetween) and a pitch of 0.01-0.04 inches (and all values and ranges therebetween) and a thread angle of 30-70° (and all values and ranges therebetween). Head threading 230 is illustrated as being a single thread. The pitch of the body threading 260 is generally greater than the pitch of bead threading 230 (e.g., at least 20% greater; 20-80% greater an all values and ranges therebetween). In one non-limiting configuration pitch of body threading 260 is 0.05-0.12 inches. The lead of body threading 260 is generally greater than the lead of head threading 430 (e.g., at least 20% greater; 20-80% greater an all values and ranges therebetween). In one non-limiting configuration lead of body threading 260 is 0.05-0.12 inches.

As illustrated in FIG. 11, body threading 260 on the body portion of the orthopedic screw has a maximum cross-sectional area that is less than the maximum cross-sectional area of head threading 230.

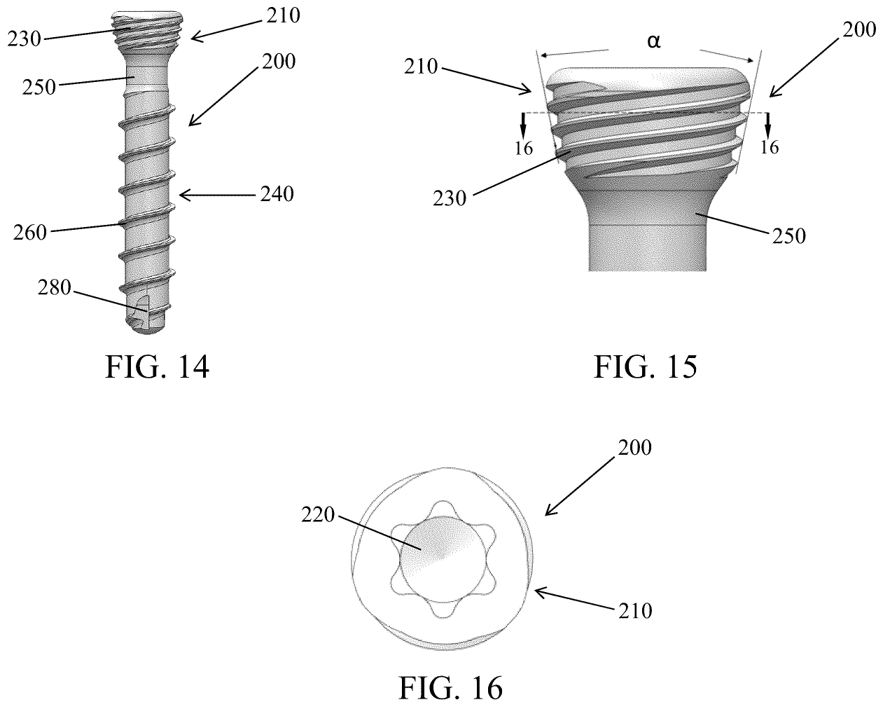

The bottom portion of body portion 240 optionally includes a cut face 280 that can be used to facilitate in the tip of orthopedic screw 200 penetrating into a bone. As illustrated in FIG. 14, the cut face 280 intersects a portion of body threading 260 thereby forming a break in the body threading 260 at the bottom portion of body portion 240. As illustrated in FIG. 14, only the most bottom portion of the body threading 260 is intersected by the cut face 280. The cut face 280 generally encircles no more than 30% (e.g., 2-30% and all values and ranges therebetween) of the outer perimeter of the bottom portion of body portion 240.

Turning now to the methods of implantation, the surgeon accesses the surgical site of interest, which can be an internal site at which a bone fracture that requires stabilization to ensure proper healing is located. The fracture may be reduced with conventional forceps and guides (which are known to those in the art), and a medical device of appropriate size and shape is placed over the fracture site. In some instances, the medical device may be temporarily secured to the bone using provisional fixation pins. When used, provisional fixation pins may be used through any of the openings in the medical device. Provisional fixation pins temporarily secure the medical device to the bone before placing the fastener system (e.g., orthopedic screws, etc.) through the opening in the medical device. Thus, the surgeon can be certain that the medical device is properly positioned before placing the orthopedic screws for permanent fixation of the medical device to the bone.

Once the medical device is secured at a desired location in relation to the fracture, the surgeon then identifies an insertion angle, or the direction along which the orthopedic screws are to be inserted through one or more openings in the medical device and then driven into the bone material of the bone. If the medical device includes more than one opening, the surgeon also selects the specific openings in the medical device to be used. After selecting the desired insertion angle and openings to be used in the medical device, the surgeon inserts a lower portion of the orthopedic screw through each of the selected openings in the medical device until the bottom of the orthopedic screw contacts bone material. In some cases, a hole may need to be drilled or tapped into the bone along the insertion angle to facilitate the initial tapping or insertion of the orthopedic screw into the bone. The surgeon then uses an appropriate driving tool in the cavity of the head portion of the orthopedic screw to manipulate the orthopedic screw into place.

The orthopedic screw 100, 200 can optionally be inserted at angles other than being aligned with the central axis of opening SO in medical device MD. In some instance, the surgeon may need to toggle or maneuver orthopedic screw 100, 200 in order to secure and draw in displaced bone fragments during the securing of medical device MID to bone B.

Once the bone fragment is secured, orthopedic screw 100, 200 is ready to be secured to medical device MD. As orthopedic screw 100, 200 is driven further into bone B, it is also drawn further into medical device MD.

In one optional procedure, once all orthopedic screws 100, 200 are inserted into the medical device MD, the surgeon may optionally place covers over unused openings SO in medical device MD. Additionally or alternatively, the surgeon may use bone graft material, bone cement, bone void filler, and any other material to help heal bone B. Such material (when used) can be placed in one or more unused openings SO in the medical device MD and/or placed one and/or about one or more portions of medical device MD.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, Applicant does not intend any of the appended claims or claim elements to invoke 35 U.S.C. 112 (f) unless the words “means for” or “step for” are explicitly used in the particular claim.

What is claimed:

1. An orthopedic screw for use with a medical device; said orthopedic screw is configured to be secured to a screw opening in the medical device; said orthopedic screw includes a head portion and a body portion that is connected to the bottom of said head portion and extends downwardly from said head portion; said head portion includes head threading on an outer surface of head portion; said body portion includes body threading on an outer surface of said body portion; one or more of a) a pitch of said body threading is different from a pitch of said head threading, b) a thread length of said body threading is different from a thread length of said head threading, c) a thread angle of said body threading is different from a thread angle of said head threading, d) a root depth of said body threading is different from a root depth of said head threading, and/or e) a lead of said body threading is different from a lead of said head threading; said orthopedic screw is at least partially formed of a metal alloy that includes at least 5 awt. % rhenium.

2. The orthopedic screw as defined in claim 1, wherein said head portion of said orthopedic screw includes a tapered portion that forms a tapered profile of said head portion along a central axis of said head portion; said head threading is at least partially located on said tapered portion; said tapered portion constitutes 20-100% of a longitudinal length of said head portion.

3. The orthopedic screw as defined in claim 2, wherein an angle of taper of said tapered portion is constant along 60-100% of said tapered portion.

4. The orthopedic screw as defined in claim 2, wherein said head threading on said tapered portion also has a tapered profile.

5. The orthopedic screw as defined in claim 3, wherein said head threading on said tapered portion also has a tapered profile.

6. The orthopedic screw as defined in claim 1, wherein said body portion includes a tapered transition region that terminates at said head portion; said transition region absent threading.

7. The orthopedic screw as defined in claim 5, wherein said body portion includes a tapered transition region that terminates at said head portion; said transition region absent threading.

8. The orthopedic screw as defined in claim 1, wherein a bottom portion of said body portion includes a cut face; said cut face intersects a portion of said body threading thereby forming a break in said body threading.

9. The orthopedic screw as defined in claim 7, wherein a bottom portion of said body portion includes a cut face; said cut face intersects a portion of said body threading thereby forming a break in said body threading.

10. The orthopedic screw as defined in claim 1, wherein a top surface of said head portion has a non-circular profile; said top surface of said head portion includes a plurality of curvilinear regions having different radii.

11. The orthopedic screw as defined in claim 9, wherein a top surface of said head portion has a non-circular profile; said top surface of said head portion includes a plurality of curvilinear regions having different radii.

12. An orthopedic screw for use with a medical device; said orthopedic screw is configured to be secured to a screw opening in the medical device; said orthopedic screw has a cross-sectional area of the root that is at least 10% less that a cross-sectional area a similar shaped orthopedic screw formed of stainless steel, cobalt chromium alloy, or titanium alloy; said orthopedic screw has i) the same or greater tensile strength as compared to a similar shaped an orthopedic screw formed of stainless steel, cobalt chromium alloy, or titanium alloy, ii) the same or greater ductility as compared to a similar shaped an orthopedic screw formed of stainless steel, cobalt chromium alloy, or titanium alloy, iii) a reduced corrosion rate as compared a similar shaped an orthopedic screw formed of stainless steel, cobalt chromium alloy, or titanium alloy, iv) a reduced ion release rate as compared to a similar shaped an orthopedic screw formed of stainless steel, cobalt chromium alloy, or titanium alloy, v) the same or greater hardness as compared to a similar shaped an orthopedic screw formed of stainless steel, cobalt chromium alloy, or titanium alloy, and/or vi) the same or greater yield strength as compared to a similar shaped an orthopedic screw formed of stainless steel, cobalt chromium alloy, or titanium alloy; said orthopedic screw is at least partially formed of a metal alloy that includes at least 5 awt. % rhenium.

13. A orthopedic screw for use with a medical device, said orthopedic screw is configured to be secured to a screw opening in the medical device; a thread on a head portion of said orthopedic screw has a cross-sectional area that is at least 10% less than a cross-sectional area of a thread on a head portion of a similar shaped orthopedic screw formed of stainless steel, cobalt chromium alloy, or titanium alloy; said orthopedic screw has i) the same or greater tensile strength as compared to a similar shaped an orthopedic screw formed of stainless steel, cobalt chromium alloy, or titanium alloy, ii) the same or greater ductility as compared to a similar shaped an orthopedic screw formed of stainless steel, cobalt chromium alloy, or titanium alloy, iii) a reduced corrosion rate as compared a similar shaped an orthopedic screw formed of stainless steel, cobalt chromium alloy, or titanium alloy, iv) a reduced ion release rate as compared to a similar shaped an orthopedic screw formed of stainless steel, cobalt chromium alloy, or titanium alloy, v) the same or greater hardness as compared to a similar shaped an orthopedic screw formed of stainless steel, cobalt chromium alloy, or titanium alloy, and/or vi) the same or greater yield strength as compared to a similar shaped an orthopedic screw formed of stainless steel, cobalt chromium alloy, or titanium alloy; said orthopedic screw is at least partially formed of a metal alloy that includes at least 5 awt. % rhenium.

14. A orthopedic screw for use with a medical device, said orthopedic screw is configured to be secured to a screw opening in the medical device; a thread on the body portion of the orthopedic screw has a cross-sectional area that is at least 10% less than a cross-sectional area of a thread on the body portion of a similar shaped orthopedic screw formed of stainless steel, cobalt chromium alloy, or titanium alloy; said orthopedic screw has i) the same or greater tensile strength as compared to a similar shaped an orthopedic screw formed of stainless steel, cobalt chromium alloy, or titanium alloy, ii) the same or greater ductility as compared to a similar shaped an orthopedic screw formed of stainless steel, cobalt chromium alloy, or titanium alloy, iii) a reduced corrosion rate as compared a similar shaped an orthopedic screw formed of stainless steel, cobalt chromium alloy, or titanium alloy, iv) a reduced ion release rate as compared to a similar shaped an orthopedic screw formed of stainless steel, cobalt chromium alloy, or titanium alloy, v) the same or greater hardness as compared to a similar shaped an orthopedic screw formed of stainless steel, cobalt chromium alloy, or titanium alloy, and/or vi) the same or greater yield strength as compared to a similar shaped an orthopedic screw formed of stainless steel, cobalt chromium alloy, or titanium alloy; said orthopedic screw is at least partially formed of a metal alloy that includes at least 5 awt. % rhenium.
